(19) United States
(12) Patent Application Publication
(10) Pub. No.: US 2023/0417754 A1
(43) Pub. Date: Dec. 28, 2023

(54) NEAR INFRARED-II PROBES AS HIGH AFFINITY TARGETING IMAGING AGENTS AND USES THEREOF

(71) Applicant: On Target Laboratories, LLC, West Lafayette, IN (US)

(72) Inventor: Sumith A. Kularatne, West Layfayette, IN (US)

(21) Appl. No.: 18/252,904

(22) PCT Filed: Nov. 16, 2021
(86) PCT No.: PCT/US2021/059529

§ 371 Date: May 15, 2023

Related U.S. Application Data
(60) Provisional application No. 63/115,132, filed on Nov. 18, 2020.

(51) Int. Cl. G01N 33/574; C09B 23/01; A61K 49/00; G01N 33/58

(52) U.S. Cl. CPC G01N 33/57492; C09B 23/0066; A61K 49/0032; A61K 49/0056; G01N 33/582; G01N 2800/52; G01N 2800/54

(57) ABSTRACT

The present disclosure relates to methods and compositions for detecting a target cell using a compound comprising a targeting moiety, a linker, and a NIR-II dye.

### CROSS-REFERENCE TO RELATED APPLICATION

The present application claims priority to U.S. Provisional Application No. 63/115,132, filed Nov. 18, 2020, which is incorporated by reference herein in its entirety.

### BACKGROUND

Surgical removal of malignant disease constitutes one of the most common and effective therapeutic for primary treatment for cancer. Resection of all detectable malignant lesions results in no detectable return of the disease in approximately 50% of all cancer patients¹ and may extend life expectancy or reduce morbidity for patients in whom recurrence of the cancer is seen ²⁻⁵. Not surprisingly, surgical methods for achieving more quantitative cytoreduction are now receiving greater scrutiny.

Given the importance of total resection of the malignant lesions, it is extremely important to ensure that the malignant lesions are accurately and completely identified. Identification of malignant tissue during surgery is currently accomplished by three methods. First, many tumor masses and nodules can be visually detected based on abnormal color, texture, and/or morphology. Thus, a tumor mass may exhibit variegated color, appear asymmetric with an irregular border, or protrude from the contours of the healthy organ. A malignant mass may also be recognized tactilely due to differences in plasticity, elasticity or solidity from adjacent healthy tissues. Finally, a few cancer foci can be located intraoperatively using fluorescent dyes that flow passively from the primary tumor into draining lymph nodes⁶⁻⁹. In this latter methodology, fluorescent (sentinel) lymph nodes can be visually identified, resected and examined to determine whether cancer cells have metastasized to these lymph nodes.

Fluorescence imaging technology has gradually become a new and promising tool for in vivo visualization detection. Because it can provide real-time sub-cellular resolution imaging results, it can be widely used in the field of biological detection and medical detection and treatment. Conventional fluorescent techniques use probes in the visible light spectrum (˜400-700 nm),¹⁰ which is not optimal for intra-operative image-guided surgery as it is associated with a relatively high level of nonspecific background light due to collagen in the tissues. Hence, the signal to noise ratio from these conventional compounds is low. Moreover, the absorption of visible light by biological chromophores, in particular hemoglobin, limits the penetration depth to a few millimeters (1-2 mm).¹⁰ Thus, tumors that are buried deeper than a few millimeters in the tissue may remain undetected. Moreover, ionization equilibrium of fluorescein (pKa=6.4) leads to pH-dependent absorption and emission over the range of 5 to 9. Therefore, the fluorescence of fluorescein-based dyes is quenched at low pH (below pH 5). Due to the limited imaging depth (1-2 mm) and self-fluorescence background of tissue emitted in the visible region (400-700 nm), it fails to reveal biological complexity in deep tissues.

The traditional near infrared wavelength (NIR-I, 650-950 nm),¹⁰ which is considered as the first biological or optical window, because it reduces the NIR absorption and scattering from blood and water in organisms. Because near-infrared light (NIR) is less absorbed and scattered in biological tissues than visible light, the former can penetrate biological tissues, such as skin, more effectively. The penetration of the NIR-I fluorescence bioimaging is larger (1-2 cm) than that of visible light (1-2 mm). In fact, NIR-I fluorescence bioimaging is still interfered by tissue autofluorescence (background noise), and the existence of photon scattering, which limits the depth of tissue penetration. Recent experimental and simulation results show that the signal-to-noise ratio (SNR) of bioimaging can be significantly improved at the second region near infrared (NIR-II, 1,000-1,700 nm), also known as the second biological window.¹⁰ NIR-II bioimaging is able to explore deep-tissues information in the range of centimeter, and to obtain micron-level resolution at the millimeter depth, which surpass the performance of NIR-I fluorescence imaging. The key of fluorescence bioimaging is to achieve highly selective imaging. Therefore, compared with visible light (400-700 nm) and traditional NIR-I window (650-950 nm), NIR-II window (950-1,700 nm) can avoid background interference such as spontaneous fluorescence and photon scattering, 10 thereby able to image deep tissue (1-7 cm).

Despite the recognition of the importance of removal of tumor and the availability of certain identification techniques for visualizing tumor mass, many malignant nodules still escape detection, leading to disease recurrence and often death¹⁰⁻¹³. Thus, there is a need for improved tumor identification. This motivation has led to introduction of two new approaches for intraoperative visualization of malignant disease. In the first, a quenched fluorescent dye is injected systemically into the tumor-bearing animal, and release of the quenching moiety by a tumor-specific enzyme, pH change, or change in redox potential is exploited to selectively activate fluorescence within the malignant mass.¹¹⁻¹⁷ In the second approach, a fluorescent dye is conjugated to a tumor-specific targeting ligand that causes the attached dye to accumulate in cancers that over-express the ligand's receptor. Examples of tumor targeting ligands used for this latter purpose include folic acid, which exhibits specificity for folate receptor (FR) positive cancers of the ovary, kidney, lung, endometrium, breast, and colon²¹⁻²², DUPA, which can deliver attached fluorescent dyes selectively to cells expressing prostate-specific membrane antigen (PSMA), i.e. prostate cancers and the neovasculature of other solid tumors¹⁸⁻²⁰, and CBA, which selectively deliver attached dyes to cells expressing carbonic anhydrase nine (CA IX) receptor (i.e. Kidney and colon cancer cells) and tumor microenvironment expressing CA IX under acidosis.

Thus, there remains a need for a dye substance that can be used to specifically target deep diseased tissue and has increased stability and brightness for use in vivo for tissue imaging.

### SUMMARY

One aspect of the present technology targeted ligands linked to NIR-II dyes via different linkers.

In an effort to overcome drawbacks associated with tumor detection, complete resection of tumors with negative tumor margins, and the inventors developed novel tumor-targeted low molecular weight NIR-II dyes [i.e. folate-, PSMA-, carbonic anhydrase nine- (CA IX), glucose transporter one- (Glut1), fibroblast activating protein- (FAP), cholecystokinin B receptor- (CCK2R), etc. targeted NIR-II dyes). Each tumor-targeted NIR-II demonstrated a very high affinity and specificity for the requisite biomarker/receptor/protein that is overexpressed diseased cell such as cancer and inflammatory cells. Moreover, when standard NIR-II dyes are employed as a ligand-targeted fluorescent probe, no toxicity is generally observed and the emitted fluorescence can often be detected up to 7-10 cm beneath the tissue surface or skin.

In one aspect of the present technology, the compounds of the present invention have the form: B-L-X, wherein B is a tumor-targeted ligand, L is a linker, and X is a NIR-II dye.

Another aspect of the present technology is a method of identifying a target cell in a biological sample using compounds of the present invention having the form B-L-X, wherein B is a tumor-targeted ligand, L is a linker, and X is a NIR-II dye. In some aspects, the steps of this method include contacting a biological sample with a compound of the form B-L-X for a time and under conditions sufficient for binding of the compound to the target cell and optically detecting the presence or absence of the compound in the biological sample; wherein presence of the compound in detecting step indicates that the target cell is present in the biological sample.

Another aspect of the present technology is a method of performing image-guided surgery on a subject using compounds of the present invention having the form B-L-X, wherein B is a tumor-targeted ligand, L is a linker, and X is a NIR-II dye. The method comprises the steps of administering a compound of the formula B-L-X to the subject for a time and under conditions sufficient for the compound to accumulate at a surgical site of the subject; illuminating the surgical site to visualize the compound using near infrared light; and performing surgical resection of tissue that fluoresces upon excitation with the near infrared light.

Another aspect of the present technology is a method of diagnosing a disease in a subject using compounds of the present invention having the form B-L-X, wherein B is a tumor-targeted ligand, L is a linker, and X is a NIR-II dye. The method comprises the steps of administering a compound of the formula B-L-X to the subject for a time and under conditions sufficient for binding of the compound to a target cell in a tissue of the subject; illuminating the tissue to visualize the compound using near infrared light; measuring a fluorescent signal from the compound upon excitation with the near infrared light; comparing the fluorescent signal measured in the previous step with at least one control data set, wherein the at least one control data set comprises a fluorescent signal from the compound of the formula B-L-X contacted with a biological sample that does not comprise the target cell; and diagnosing the subject with the disease, wherein the comparison in the previous step indicates the presence of the disease.

Another aspect of the present technology are methods for detecting circulating tumor cells (CTCs) in a subject using a compound of the present invention having the form B-L-X, wherein B is a tumor-targeted ligand, L is a linker, and X is a NIR-II dye. In some aspects, the method comprises contacting a bodily fluid of the subject with the compound for a time that allows for binding of the compound to at least one CTC of a target cell type, illuminating the CTCs with an excitation light of a wavelength that is absorbed by the compound, and detecting the optical signal emitted by the compound.

In some aspects, the subject is a mammal. In other aspects, the subject is a human. In some aspects, the subject has cancer. In another aspect, the cancer is early-stage cancer or metastatic cancer.

In some aspects, the CTCs are shed from a tumor. In another aspect, the tumor is a primary tumor.

In some aspects, the detection of the CTCs is conducted ex vivo. In yet another aspect, the ex vivo detection is CTCs in bodily fluids. In a further aspect, the bodily fluid is blood.

In some aspects, the detection of the CTCs is conducted in vivo. In a further aspect, this in vivo detection can be completed in real-time. In another aspect, the method is used to track and analyze the distribution and the phenotype of cancer cells. In a further aspect, the information is tracked through a software platform. In yet another aspect, the information tracked is delivered to a smartphone and/or smartwatch app.

In some aspects, the CTCs are further quantified after detection. In one aspect, flow cytometry is used to quantitate the CTCs.

One aspect of the present technology is a method for diagnosing a disease in a subject, wherein the method comprises the detection of CTCs in the subject using a compound of the present invention having the form B-L-X, wherein B is a tumor-targeted ligand, L is a linker, and X is a NIR-II dye. In some aspects, the disease is cancer. In some aspects, the method comprises contacting a bodily fluid of the subject with the compound for a time and under conditions that allow for binding of the compound to at least one CTC, illuminating the CTCs with an excitation light of a wavelength that is absorbed by the compound, detecting the optical signal emitted by the compound, comparing the optical signal measured in the previous step with at least one control data set, wherein the at least one control data set comprises a fluorescent signal from the compound contacted with a biological sample that does not comprise CTCs, and diagnosing the subject is based on the previous step.

One aspect of the present technology is a method for detecting CTCs to provide real-time monitoring, screening, and management of subject having a disease, wherein the method comprises the detection of CTCs using a compound of the present invention having the form B-L-X, wherein B is a tumor-targeted ligand, L is a linker, and X is a NIR-II dye. In some aspects, the method comprises contacting a bodily fluid of the subject with the compound for a time and under conditions that allow for binding of the compound to at least one CTC, illuminating the CTCs with an excitation light of a wavelength that is absorbed by the compound, and detecting the optical signal emitted by the compound. In some aspects, the disease is cancer. In a further aspect, the real-time monitoring, screening, and management is tracked through a software platform. In yet another aspect, the information tracked is delivered to a smartphone and/or smartwatch app.

### BRIEF DESCRIPTION OF THE DRAWINGS

The above-mentioned and other features of this disclosure, and the manner of attaining them, will become more apparent, and the disclosure itself will be better understood by reference to the following description of embodiments of the disclosure taken in conjunction with the accompanying drawings.

FIGS. 1A and 1B are chemical structures of several NIR-II dyes.

FIGS. 2A-2S are chemical structures of several folate receptor-targeted NIR-II imaging agents.

FIGS. 3A-3L are chemical structures of several PSMA-targeted NIR-II imaging agents.

FIGS. 4A-4F are chemical structures of several CA IX-targeted NIR-II imaging agents.

### DETAILED DESCRIPTION

It is to be understood that this invention is not limited to the particular methodology, protocols, cell lines, constructs, and reagents described herein and as such may vary. It is also to be understood that the terminology used herein is for the purpose of describing particular aspects only, and is not intended to limit the scope of the present invention, which will be limited only by the appended claims.

As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The terms “functional group”, “active moiety”, “activating group”, “leaving group”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission.

In some aspects of the invention, the compounds can be used for image guided surgery, tumor imaging, lymph node imaging, inflammatory diseases, atherosclerosis, infection diseases, forensic applications, mineral applications, dental, gel staining, DNA sequencing, nerve staining, or plastic surgery.

In some aspects of this invention, the subject can be any mammalian subject, including, but not limited to a human subject. In some aspects, the compound is in the form a pharmaceutically acceptable salt. Pharmaceutically acceptable salts include, but are not limited to, sodium, potassium, ammonium, calcium, magnesium, lithium, cholinate, lysinium, and hydrogen salts. In some aspects, the compound is formulated as composition. The composition may be pharmaceutically or therapeutically acceptable. In other aspects, the composition may comprise a pharmaceutically or therapeutically acceptable amount of the compound.

In some aspect of the invention, the compound may be incorporated into targeting moieties which may include a protein or polypeptide, such as an antibody, or biologically active fragment thereof, preferably a monoclonal antibody, small molecules, aptamers, DNA, or RNA. The supplemental fluorescing targeting construct(s) used in practice of the disclosure method may also be or comprise polyclonal or monoclonal antibodies tagged with a fluorophore. The term “antibody” as used in this disclosure includes intact molecules as well as functional fragments thereof, such as Fab, F(ab′)2, and Fv that are capable of binding the epitopic determinant. Methods of making these fragments are known in the art. (See for example, Harlow & Lane, Antibodies: A Laboratory Manual, Cold Spring Harbor Laboratory, New York, 1988, incorporated herein by reference). As used in this disclosure, the term “epitope” means any antigenic determinant on an antigen to which the paratope of an antibody binds. Epitopic determinants usually consist of chemically active surface groupings of molecules such as amino acids or sugar side chains, and usually have specific three-dimensional structural characteristics, as well as specific charge characteristics.

In some aspects, a compound may be incorporated or used with other fluorescing targeting constructs (e.g., antibodies, or biologically active fragments thereof, having attached fluorophores) that bind to other receptors or antigens on the tumor or tissue (e.g., a site of atherosclerosis, infection, cardiovascular disease, neurodegenerative disease, immunologic disease, autoimmune disease, respiratory disease, metabolic disease, inherited disease, infectious disease, bone disease, and environmental disease or the like) to be imaged. Any additional targeting moiety that specifically targets the tumor or specific site on the tissue may be used provided that it is specific for the site to be monitored. The purpose of the additional fluorescing targeting construct is to increase the intensity of fluorescence at the site to be monitored thereby aiding in detection of diseased or abnormal tissue in the body part. For example, a given tumor may have numerous markers and in addition to the compounds of the present disclosure a cocktail of fluorescent moieties is provided which are specific for that given tumor such that the signal emanating from the tumor is generated by more than one compound or fluorescent moiety that has targeted and localized to the tumor site of interest.

In practice, the skilled person would administer a compound of the present disclosure either alone or as part of a cocktail of targeting detectable moieties and allow these compounds and targeting moieties to bind to and/or be taken up by any target tissue that may be present at the site under investigation and then provide a supply of the light source. Typically, the compounds of the present disclosure and any additional targeting moieties will be administered prior to surgery or a minimally invasive or non-invasive procedure for a time and in compositions that allow the fluorescent compounds of the present disclosure as well as any additional fluorescent constructs to be taken up by the target tissue.

Those of skill in the art will be able to devise combinations of successively administered fluorescing targeting constructs, each of which specifically binds to the target site. It is preferable that all of the fluorescing targeting constructs used in such cocktails to identify the target tissue comprise fluorophores that fluoresce within the same wavelength band or at the same wave length as does the compound of the present disclosure (e.g. a fluorescing sensitive to near infrared wavelength of light in the compounds of the present disclosure) to minimize the number of different light sources that need to be employed to excite simultaneous fluorescence from all of the different targeting constructs used in practice of the disclosure method. However, it is contemplated that the additional targeting moieties other than the compounds of the present disclosure may fluoresce in response to the irradiating light at a different color (i.e., has a different wavelength) than that from the florescent compounds of the present disclosure. The difference in the colors of the fluorescence emanating from the compounds of the present disclosure and those of the additional targeting compounds may aid the observer in determining the location and size of the diseased or target tissue. In some examples, it may be desirable to include fluorophores in targeting constructs targeted to normal tissue and the compounds of the present disclosure targeted to diseased tissue such that the contrast between the diseased tissue and normal tissue is further enhanced to further aid the observer in determining the location and size of the diseased tissue. The use of such additional fluorophores and targeting agents in addition to the compounds of the present disclosure provides the advantage that any natural fluorescence emanating from normal tissue is obscured by the fluorescence emanating from fluorophore(s) in supplemental targeting constructs targeted to the normal tissue in the body part. The greater the difference in color between the fluorescence emanating from normal and target tissue, the easier it is for the observer to visualize the outlines and size of the target tissue. For instance, targeting a fluorescing targeting construct comprising a fluorophore producing infrared light from the compounds of the present disclosure to the target tissue (i.e., abnormal tissue) and a fluorophore producing green light to healthy tissue aids the observer in distinguishing the target tissue from the normal tissue. Those of skill in the art can readily select a combination of fluorophores that present a distinct visual color contrast.

The spectrum of light used in the practice of the disclosure method is selected to contain at least one wavelength that corresponds to the predominate excitation wavelength of the targeting construct, or of a biologically compatible fluorescing moiety contained within the targeting construct.

However, when a combination of targeting ligands that fluoresce at different wavelengths is used in practice of the disclosure, the spectrum of the excitation light must be broad enough to provide at least one excitation wavelength for each of the fluorophores used. For example, it is particularly beneficial when fluorophores of different colors are selected to distinguish normal from diseased tissue, that the excitation spectrum of the light(s) includes excitation wavelengths for the fluorophores targeted to normal and target tissue.

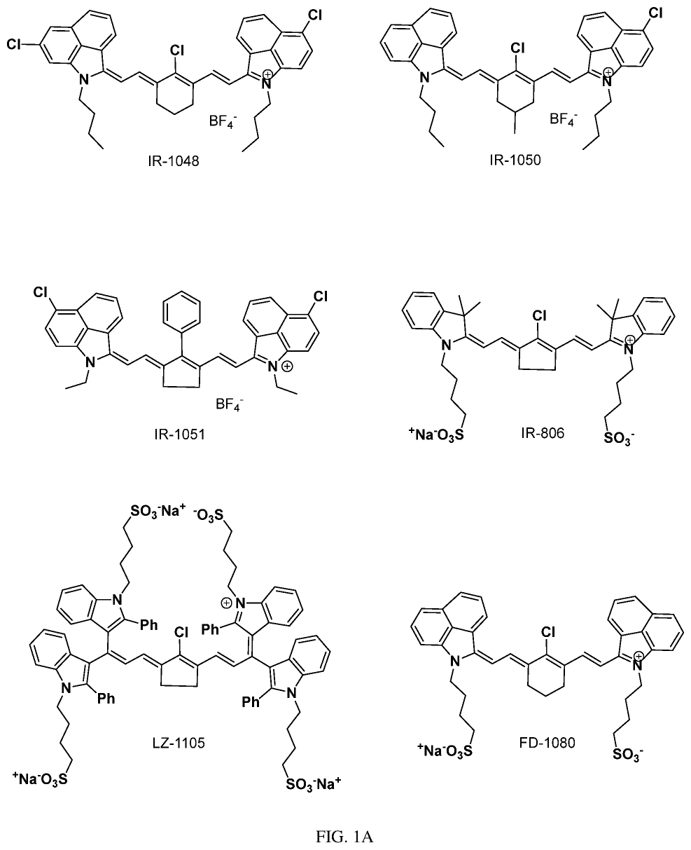

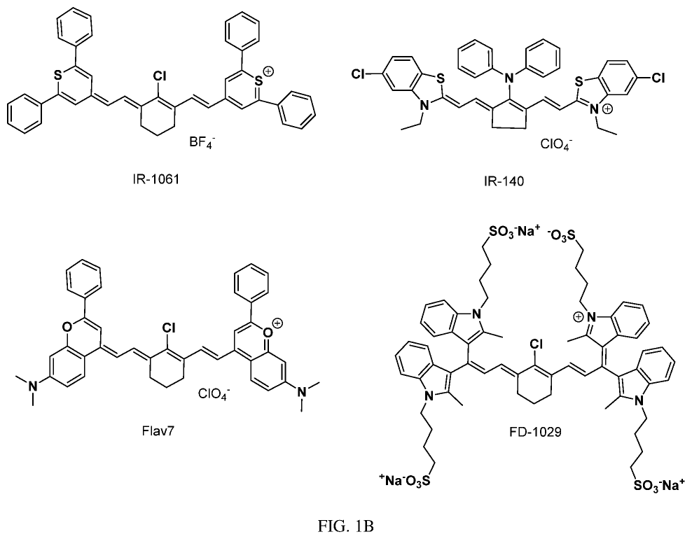

In one aspect of the present technology, the compounds of the present invention have the form: B-L-X, wherein B is a tumor-targeted ligand, L is a linker, and X is a NIR-II dye. Exemplary NIR-II dyes are shown in FIGS. 1A and 1B.

In some aspects, the tumor-targeted ligand targets a folate receptor, Glutamate carboxypeptidase II, prostate-specific membrane antigen, carbonic anhydrase IX (CA IX), Fibroblast activation protein alpha, Glucose transporter 1, or cholecystokinin-2. In some aspects, the targeting moiety is conjugated to a hydrophobic spacer or amino acid linking group. In another aspect, the targeting moiety is selected from a group comprising of a pteroyl ligand, PSMA-targeting compound, or CA IX-targeted molecule conjugated to an amino acid linking group.

In some aspects, the linker is selected from the group consisting of hydrophobic amino acids or moieties, such as neutral nonpolar (hydrophobic) amino acids, such as alanine, leucine, isoleucine, valine, proline, phenylalanine, tryptophan, and methionine or aromatic group, cyclohexyl group, tyrosine, and derivative thereof; basic (positively charged) amino acids such as arginine, histidine, and lysine and derivative thereof; neutral polar amino acids, such as glycine, serine, threonine, cysteine, tyrosine, asparagine, and glutamine and derivative thereof; In some aspects, L is an aromatic amino acid and derivative thereof. In some aspects, L has a positive charge. In other aspects, L has a negative charge.

In some aspects, L is a hydrophobic spacer. In some aspects, L is selected from the group consisting of an six aminohectanoic acid (SAHA), eight aminooctonoic acid (EAOA), polyethylene glycol (PEG), polyethylene amine (PEA) unit, a chain of 6 atoms, a spacer 6 atoms in length, a chain from 6 to 20 atoms in length; a peptide comprising aryl or aryl alkyl groups, each of which is optionally substituted, and where one aryl or aryl alkyl group is about 6 to about 10, or about 6 to about 14 atoms, and the other aryl or aryl alkyl group is about 10 to about 14, or about 10 to about 15 atoms. In another aspect, L comprises about 1 to about 20 atoms. In some aspects, the spacer is 6 atoms in length. In some aspects, the spacer comprises EAOA. In some aspects, the spacer is variably charged. In some aspects, L is peptide compromising positively charge amino acids (e.g. Arg, Lys, Orn) or quaternary amine containing amino acid. In other aspects, L has a negative charge.

In some aspects, L is selected from the group consisting polyether, a sulfonic acid and derivatives thereof, glycans and derivatives thereof, or amino acids and derivatives thereof. In some aspects, L is selected from the group consisting of the polyether is selected from the group consisting of polyethylene glycol (PEG), polyethylene oxide (PEO), or polyoxyethylene (POE). In some aspects, L is selected from the group consisting releasable linkers or non-releasable linker and derivatives thereof

In some aspects, X is variably charged. In some aspects, X has a positive charge. In other aspects, X has a negative charge. Non-limiting examples of suitable NIR-II dyes are disclosed in, for example, FIGS. 1A and 1B.

In some aspect, L is an amino acid linking group.

In some aspects, L is at least one standard amino acid. The standard amino acids include alanine, arginine, asparagine, aspartic acid, cysteine, glutamic acid, glutamine, glycine, histidine, isoleucine, leucine, lysine, methionine, phenylalanine, proline, pyrrolysine, selenocysteine, serine, threonine, tryptophan, tyrosine and valine.

In some aspects, L is at least one non-standard amino acid. Examples of non-standard amino acids include but are not limited to ornithine, homoarginine, citrulline, homocitrulline, homoserine, theanine, γ-aminobutyric acid, 6-aminohexanoic acid, sarcosine, cartinine, 2-aminoadipic acid, pantothenic acid, taurine, hypotaurine, lanthionine, thiocysteine, cystathionine, homocysteine, β-amino acids such as β-alanine, β-aminoisobutyric acid, β-leucine, β-lysine, β-arginine, β-tyrosine, β-phenylalanine, isoserine, β-glutamic acid, β-tyrosine, β-dopa (3,4-dihydroxy-L-phenylalanine), α,α-disubstituted amino acids such as 2-aminoisobutyric acid, isovaline, di-n-ethylglycine, N-methyl acids such as N-methyl-alanine, L-abrine, hydroxy-amino acids such as 4-hydroxyproline, 5-hydroxylysine, 3-hydroxyleucine, 4-hydroxyisoleucine, 5-hydroxy-L-tryptophan, cyclic amino acids such as 1-aminocyclopropyl-1-carboxylic acid, azetidine-2-carboxylic acid and pipecolic acid.

In some aspects, L is a synthetic amino acid. Examples of synthetic amino acids include but are not limited to allylglycine, cyclohexylglycine, N-(4-hydroxyphenyl)glycine, N-(chloroacetyl)glycline ester, 2-(trifluoromethyl)-phenylalanine, 4-(hydroxymethyl)-phenylalanine, 4-amino-phenylalanine, 2-chlorophenylglycine, 3-guanidino propionic acid, 3,4-dehydro-proline, 2,3-diaminobenzoic acid, 2-amino-3-chlorobenzoic acid, 2-amino-5-fluorobenzoic acid, allo-isoleucine, tert-leucine, 3-phenylserine, isoserine, 3-aminopentanoic acid, 2-amino-octanedioic acid, 4-chloro-β-phenylalanine, β-homoproline, β-homoalanine, 3-amino-3-(3-methoxyphenyl)propionic acid, N-isobutyryl-cysteine, 3-amino-tyrosine, 5-methyl-tryptophan, 2,3-diaminopropionic acid, 5-aminovaleric acid, and 4-(dimethylamino)cinnamic acid.

In further aspects, the amino acid can be tyrosine, serine, threonine, lysine, arginine, asparagine, glutamine, cysteine, selenocysteine, isomers, and the derivatives thereof. In certain aspects, the amino acid, isomers, or the derivatives thereof, contain an —OH, —NH₂, or —SH functional group that upon addition of the fluorescent dye in slight molar excess produces the conjugation of fluorescent group with the amino acid, isomer, or the derivatives thereof. In other aspects, the amino acid, isomers, or the derivatives thereof, contains an —OH functional group that upon synthesis generates an ether bond with the dye that increases the brightness and detection of the compound. In some aspects, this disclosure relates to the conjugation of the amino acid linking group with the NIR dye, wherein the amino acid, isomers, or the derivatives thereof, contains an —SH, —SeH, —PoH, or —TeH functional group that upon synthesis generates a C—S, C—Se, C—Po, or C—Te bond with the dye.

After the compound contacts the bodily fluid for a time that allows for binding of the compound to at least one target cell or CTC, the target cell or CTC(s) is illuminated with an excitation light of a wavelength that is absorbed by the compound.

In some aspects, the compounds of the preset invention have an absorption and emission maxima between about 1000 nm and about 1700 nm.

In some aspects, the compounds of the present invention are made to fluoresce after distribution thereof in cells.

In some aspects, the compound may have one or more fluorescence-imaging agents; alternatively, two more fluorescence-imaging agents, wherein each fluorescence-imaging agent has a signal property is distinguishable from the other. Those of skill in the art will be able to devise combinations of successively administered fluorescing imaging agents, each of which specifically binds to the target site. In some examples, it may be desirable to include fluorophores in targeting constructs targeted to normal cells and the compounds of the present disclosure targeted to target cells, target tissue, or CTCs such that the contrast between the target cells, target tissue, CTCs and healthy or normal cells are enhanced further to aid the observer in determining the location and size of the target cells, target tissue, or CTCs. The use of such additional fluorophores and targeting agents in addition to the compounds of the present disclosure provides the advantage that any natural fluorescence emanating from normal cells are obscured by the fluorescence emanating from fluorophore(s) in supplemental targeting constructs targeted to the normal cells. The greater the difference in color between the fluorescence emanating from normal cells and target cells, target tissue, or CTCs, the easier it is for the observer to visualize the outlines and size of the target cells, target tissue, or CTCs. Those of skill in the art can readily select a combination of fluorophores that present a distinct visual color contrast.

The spectrum of light used in the practice of the disclosed method is selected to contain at least one wavelength that corresponds to the predominate excitation wavelength of the fluorescence-imaging agent. In some aspects, the method employs laser-induced fluorescence, laser-stimulated fluorescence, or light-emitting diodes.

In one aspect, the optical signal emitted by the compound is detected. The means used to detect the compounds vary based on factors including the identity of the imaging agent, whether the method is being practiced in vitro, in vivo, or ex vivo, and when practiced in vivo, the location in the subject to be visualized. However, suitable detection methods include, but are not limited to, immunofluorescence and immunocytochemistry, FISH (fluorescence in situ hybridization), SE-iFISH (immunostaining-FISH combined with subtraction enrichment), and FACS (fluorescence assisted cell sorting). Some in vivo diagnostic imaging technologies such as computed tomography, MRI, and positron-emission tomography can detect micro-metastases to a resolution of 2-3 mm. In some aspects, to permit earlier detection of cancer, in particular metastatic cancer, an in vitro diagnostic method may be employed, which has at least a 1.5 increase in sensitivity over some in vivo methods. However, in vitro methods may be limited by the volume of bodily fluids required. Intravital, such as intravital flow cytometry, allows for the analysis of the majority of a subject's blood volume and circumvents sampling limitations and renders quantitation of rare events (<1 CTC per ml) statistically significant. Ex vivo flow cytometry allows for quantitation of small blood (e.g., 2 mL or less) and allows for further characterization and sorting.

In one aspect of the present technology, the detection of the target cells, target tissue, or CTCs is conducted ex vivo. This allows for a non-invasive or minimally invasive collection of a subject's bodily fluid(s). The term “minimally invasive”, as used herein, employs techniques that limit the size of incisions needed and so lessen wound healing time, associated pain, and risk of infection, and can include surgery. The term “non-invasive”, as used herein, refers to procedures that do not require an incision and do not break the skin to reach an intervention site. The bodily fluid includes, but is not limited to, urine, nasal secretions, nasal washes, bronchial lavages, bronchial washes, spinal fluid, sputum, gastric secretions, reproductive tract secretions (e.g., seminal fluid), lymph fluid, mucus, and blood.

In some aspects of an ex vivo method, blood samples from a cancer patient are collected. The volume of blood collected can be at least 500 μL, alternatively at least 1 mL, alternatively at least 1.5 mL, alternatively at least 2 mL, alternatively at least 2.5 mL, alternatively at least 3 mL, alternatively at least 3.5 mL, alternatively at least 4 mL, alternatively at least 4.5 mL, alternatively at least 5 mL, alternatively at least 5.5 mL, alternatively at least 6 mL, alternatively at least 6.5 mL, alternatively at least 7 mL, alternatively at least 7.5 mL, alternatively at least 8 mL, alternatively at least 8.5 mL, alternatively at least 9 mL, alternatively at least 9.5 mL, or alternatively at least 10 mL.

In some methods, the CTCs may be enriched and/or isolated using magnetic beads, buffy coat isolation, or CTC enrichment methods known in the art that incorporate the compound of the present invention or a composition that comprises the compound. After enrichment, the CTCs may be isolated by a method known in the art including, but not limited to ficoll, size-based enrichment, rosettesep, magnetophoretic mobility-based separation, microfluidic devices, fast (fiber-optic array scanning technology), flow cytometry, confocal microscopy, two-photon microscopy, epifluorescence microscopic methods.

In some aspects, the detection of the target tissue, target cells, or CTCs is conducted in vivo. An in vivo detection of target tissue, target cells, or CTCs is desirable if there are blood volume limitations with an ex vivo approach. In some aspects, a medical-grade wire or catheter is coated with a composition comprising a compound that comprises a targeting moiety and a fluorescence-imaging agent, wherein the targeting moiety targets a receptor, antigen, or antibody. In other aspects, the compound or a composition comprising the compound is administered orally, sublingually, intranasally, intraocularly, rectally, transdermally, mucosally, pulmonary, topically, or parenterally administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids).

During an in vivo procedure, the target tissue, target cells, or CTCs bind to the receptor, antigen, or antibody on the tumor targeting ligand. The bound target tissue, target cells, or CTCs are then illuminated an excitation light of a wavelength that is absorbed by the compound, and the optical signal emitted by the compound is detected. In some aspects, the compound is in contact with the bodily fluid for at least 30 minutes, alternatively at least 1 hour, alternatively at least 2 hours, alternatively at least 3 hours.

The nature of an in vivo detection is that it allows for real-time monitoring of CTCs. In some aspects, the method can be used to track and analyze the distribution and the phenotype of cancer cells. This real-time analysis may be tracked through a software platform so that a physician may actively monitor a subject's CTCs. Additionally, the software program may provide algorithms to assist in quantifying CTCs and diagnosing diseases. The algorithms may also allow for the computation of CTC trajectory and speed. The information tracked may also be provided to a subject through a smartphone and/or smartwatch app. In some aspects, the smartphone or smartwatch may provide a notification if a certain value, with respect to the CTC levels, is outside a pre-defined range.

In some methods, the CTCs are further quantified after detection. The CTCs can be quantified using techniques and methods including, but not limited to, ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods. In some aspects, flow cytometry, particularly multiphoton flow cytometry, is employed to detect and/or to quantitate the pathogenic cells.

In some in vivo methods, a compound of the present invention or a composition comprising the compound is administered to a subject with cancer. In other in vivo methods, after 1-2 hrs post-administration, CTCs can be detected using two-photon microscopy, epifluorescence microscopic, or an innovative wearable, including but not limited to, a smartwatch, a wrist band, earpiece, wearable microscope, or bicep band, that can detect the fluorescence signal.

In an exemplary embodiment, sensors and underlying algorithms are the basis for detecting and quantifying a subject's CTC levels. If an abnormal CTC level is detected, i.e., a level high or lower than a predetermined range, the subject is notified of the potential abnormality. In addition to receiving the notification, the subject can access more information related to these abnormalities on a software platform or app. Within the software platform or app, the user can see information including, but not limited to, the times when the algorithm identified an abnormality and a record of current and past CTC levels. In some embodiments, the innovative wearable, software, and/or app may be provided to a subject who has received a medical-grade wire or catheter is coated with a composition comprising a compound that comprises a targeting moiety and a fluorescence-imaging agent, wherein the targeting moiety targets a receptor, antigen, or antibody.

In another exemplary embodiment, the innovative wearable is a wearable microscope. The wearable microscope can detect and monitor CTCs labeled with the compound of the present invention. In some embodiment, the wearable microscope employs lasers to generate a fluorescent image allowing for the continuous monitoring of CTC levels. An algorithm can then process the fluorescent image, said algorithm being the basis for detecting and quantifying a subject's CTC levels. If an abnormal CTC level is detected, i.e., a level high or lower than a predetermined range, the subject is notified of the potential abnormality via the wearable microscope, software platform and/or app.

In some aspects, the compounds of the present invention are used in the imaging of cells or tissue that express a receptor, antigen, or antibody that the tumor-targeted ligand targets. In some aspects, the cells are tumor cells. In some aspects, cells are non-prostate cancer cells. In certain aspects, the cells are prostate tumor cells. In certain aspects, the cells are cancer cells. In some aspects, the present invention is used for detection of metastatic disease. In some aspects, compounds of the present invention are used for improved surgical resection and/or improved prognosis. In some aspects, methods of the present invention provide cleaner surgical margins than non-NIR conjugated fluorescing dyes. In some aspects, the NIR-II dye compounds of the present invention have an improved tumor-to-background ratio.

In other aspects, compounds of the present invention are used to image, diagnose, or detect cancer cells chosen from the group consisting of bladder cancer cells, pancreatic cancer cells, liver cancer cells, lung cancer cells, kidney cancer cells, sarcoma cells, breast cancer cells, brain cancer cells, neuroendocrine carcinoma cells, colon cancer cells, testicular cancer cells, prostate cancer cells, or melanoma cells. In other aspects, the cells being detected are more than 5 mm below the skin. In other aspects, the tumor being detected is more than 5 mm below the skin. In some aspects, the cells being detected are more than 6 mm, 7 mm, 8 mm, 9 mm, or 10 mm below the subject's skin.

In still another aspect of the methods provided, the cancer is prostate, bladder cancer, pancreatic cancer, liver cancer, lung cancer, kidney cancer, sarcoma, breast cancer, brain cancer, neuroendocrine carcinoma, colon cancer, testicular cancer or melanoma.

In some aspects, the CTCs are from a cancerous tumor, specifically a primary tumor. In some aspects, the cancer is selected from the group consisting of pancreatic, gastrointestinal, stomach, colon, ovarian, cervical, prostate, glioma, carcinoid, or thyroid, lung cancer, bladder cancer, liver cancer, kidney cancer, sarcoma, breast cancer, brain cancer, testicular cancer or melanoma.

In certain aspects, the CTCs are characterized by an intact, viable nucleus. In other aspects, the CTCs lack EpCAM or cytokeratins or are cytokeratin-positive and CD45-negative. In yet another aspect, the traditional CTCs that are undergoing apoptosis (programmed cell death). In some methods, these apoptotic CTCs may be used for monitoring a response to treatment. In some aspects, this response can be monitored in real-time. In some aspects, the CTCs are in clusters, which are two or more individual CTCs bound together.

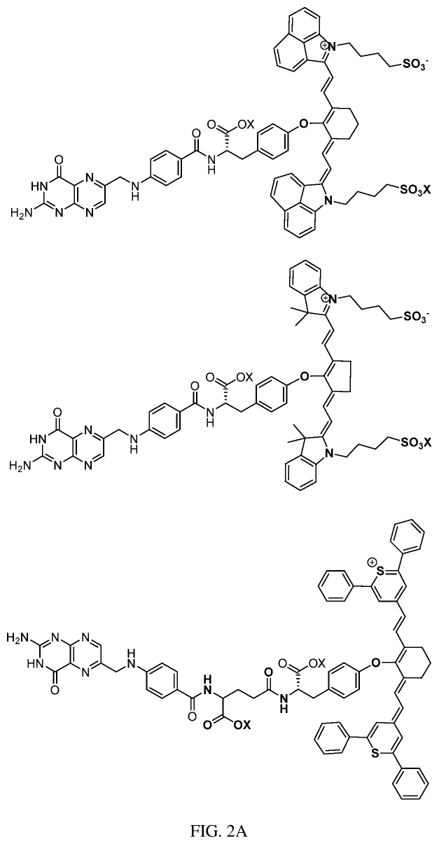

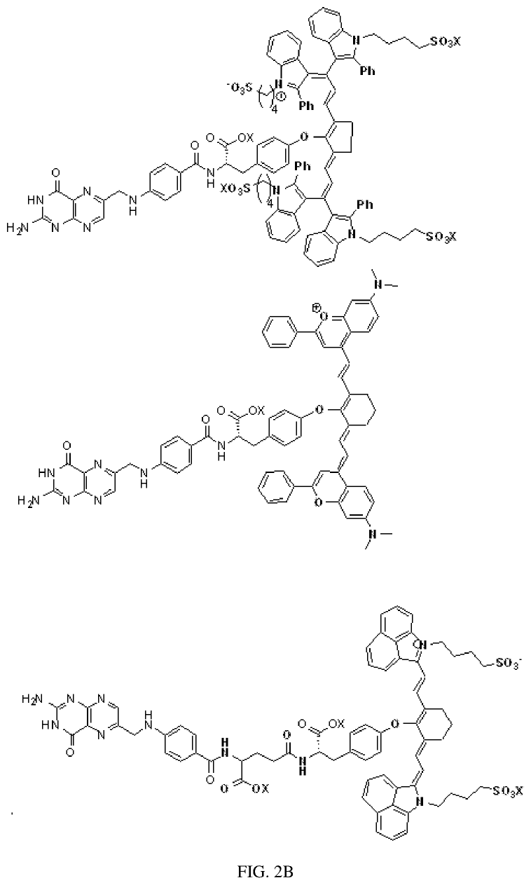

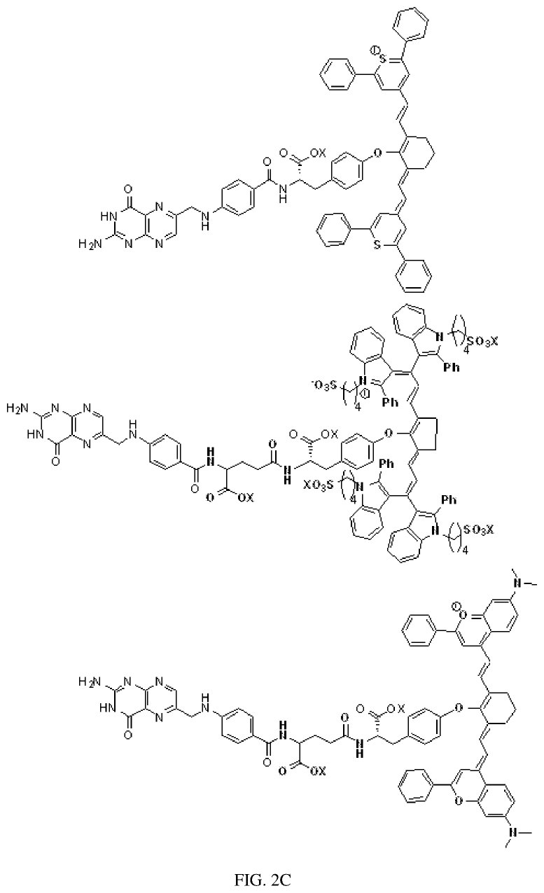

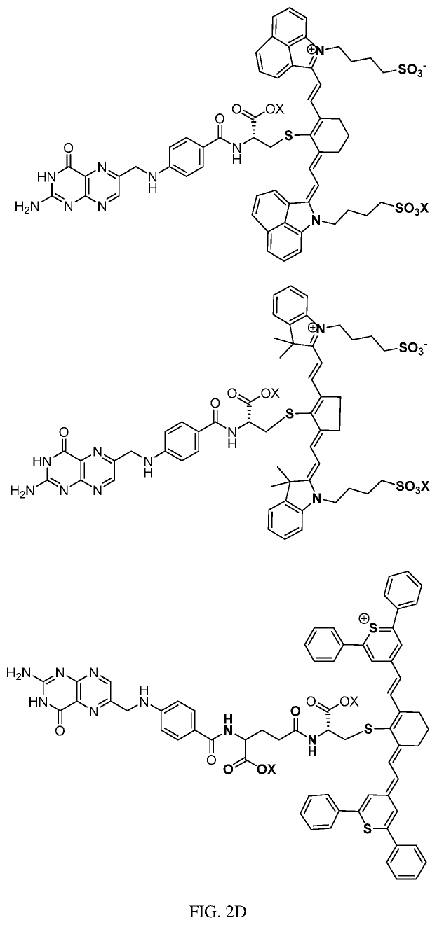

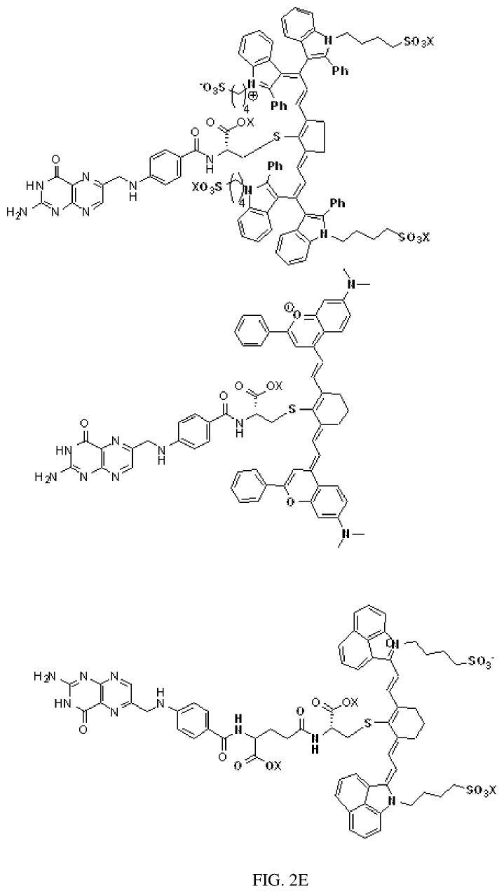

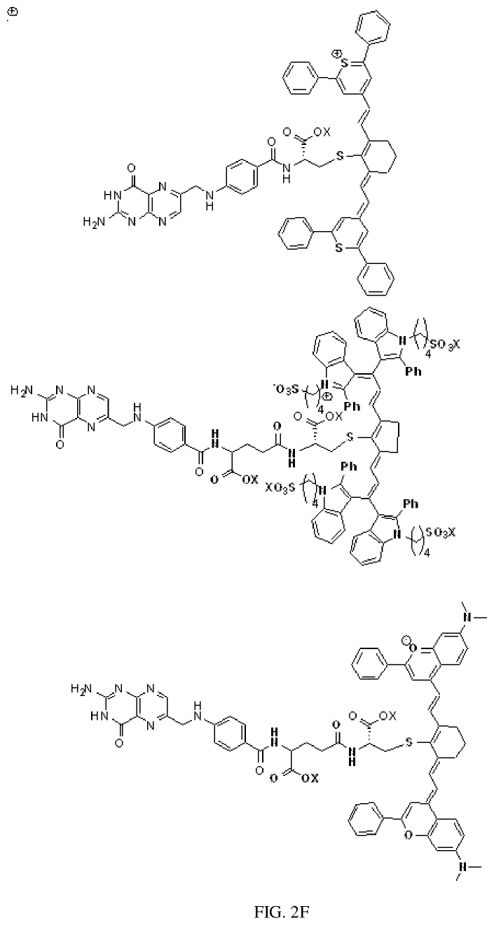

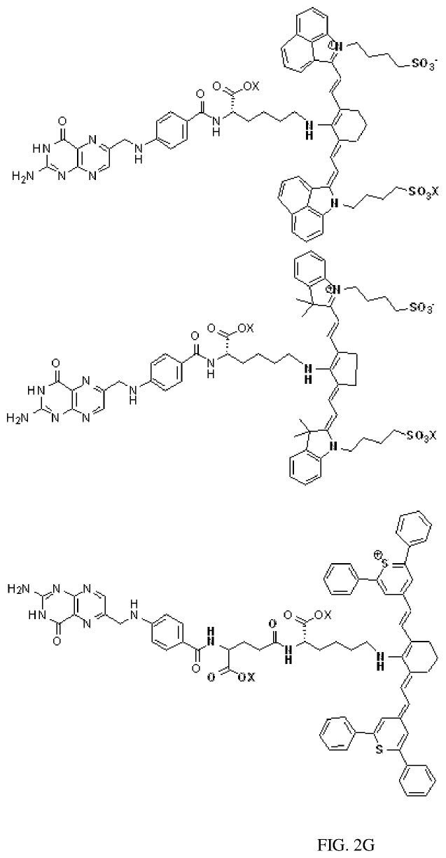

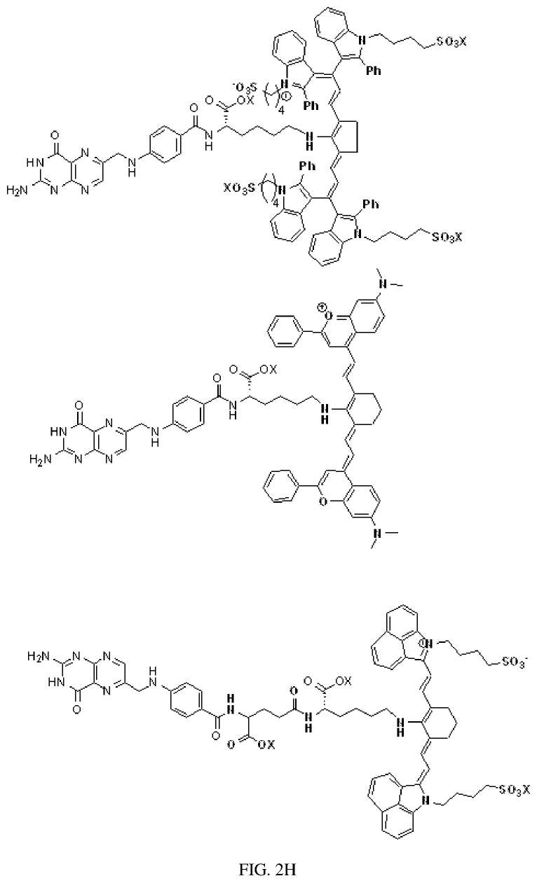

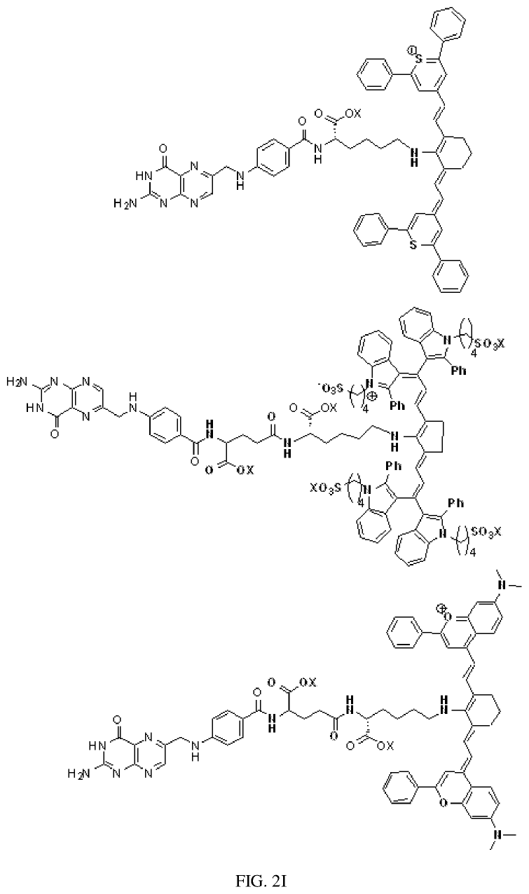

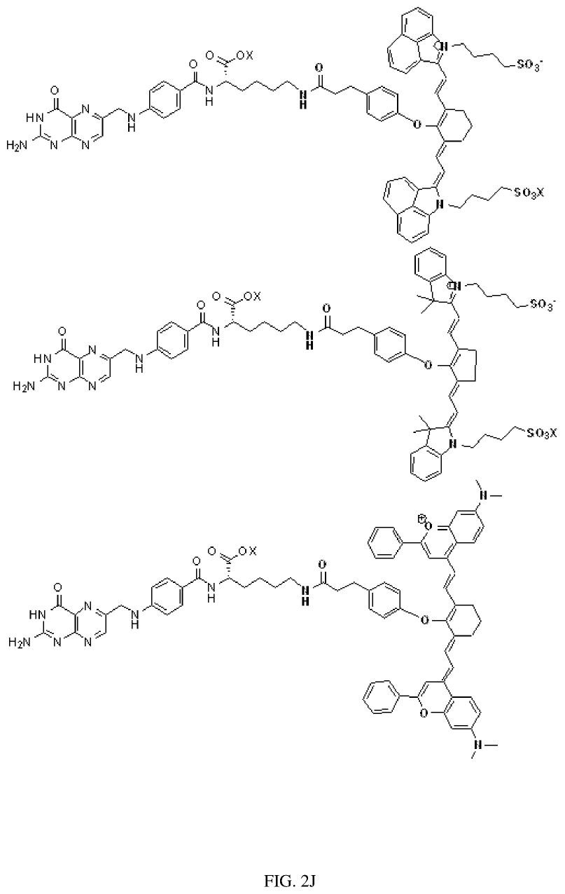

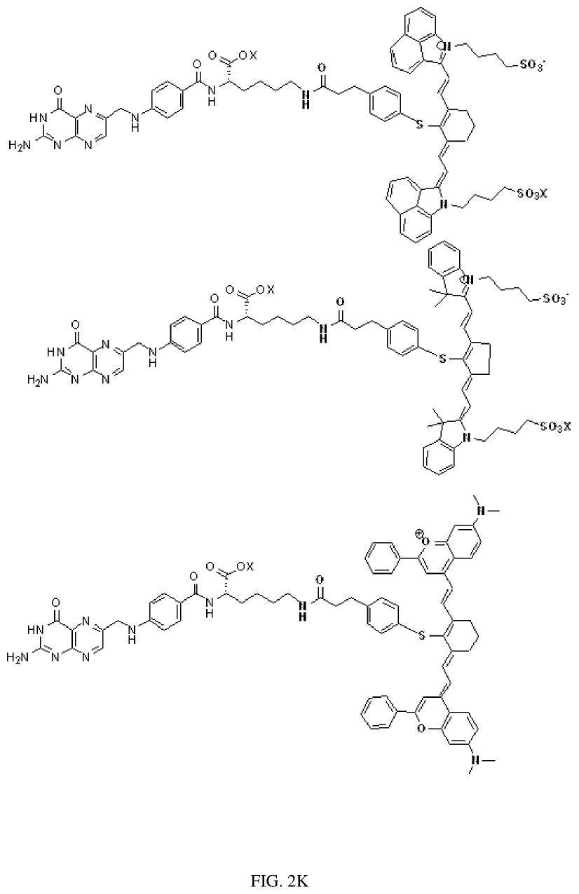

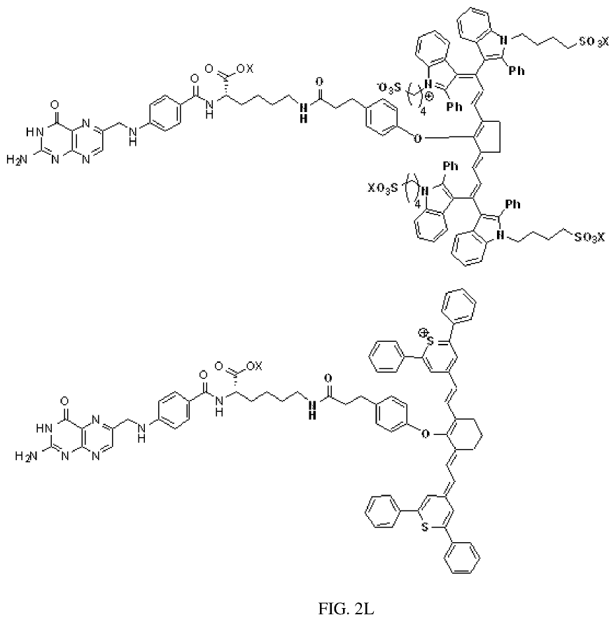

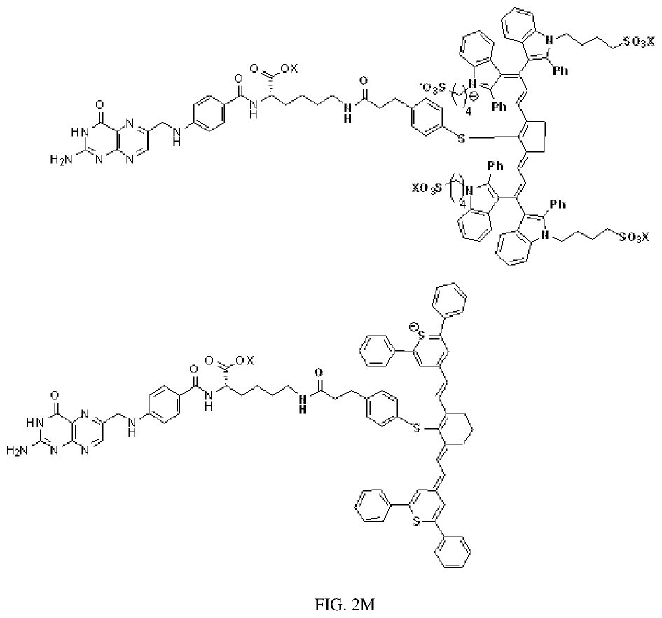

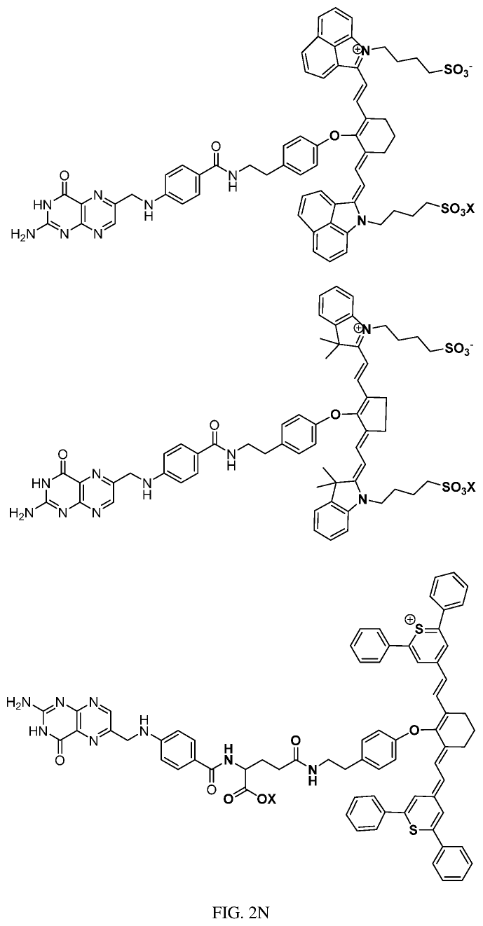

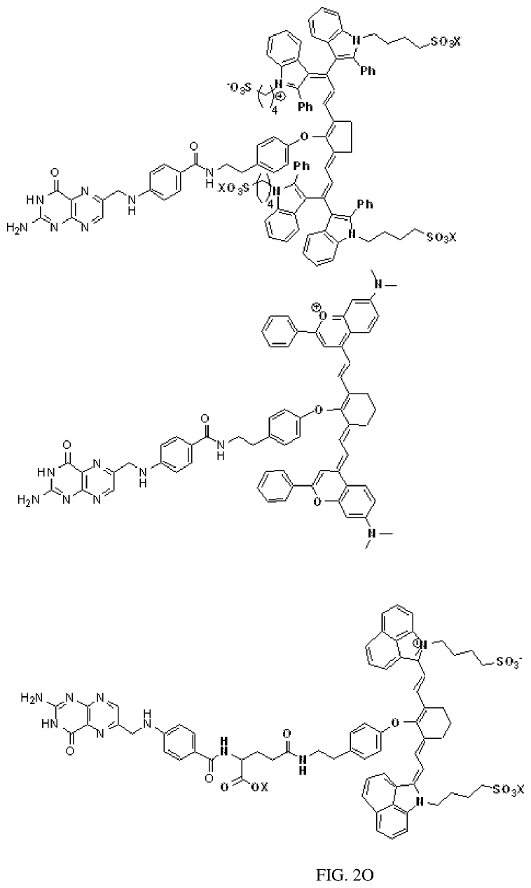

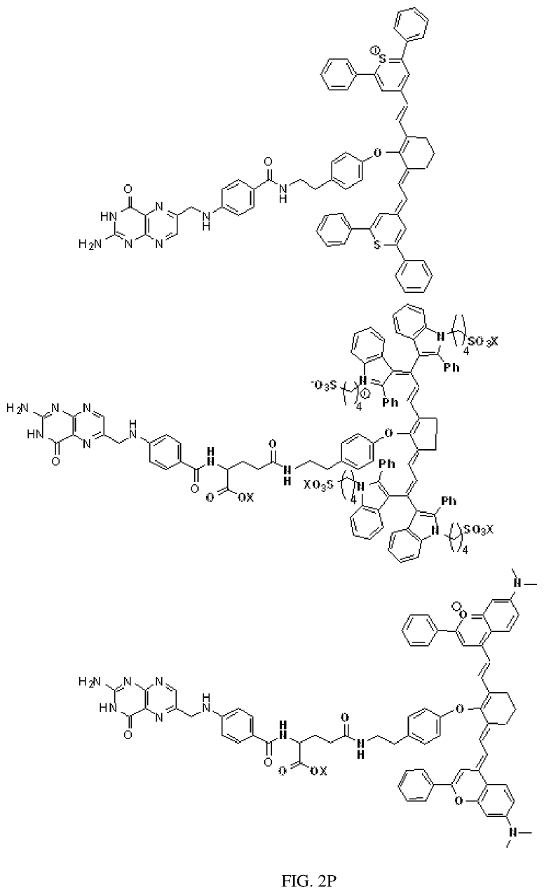

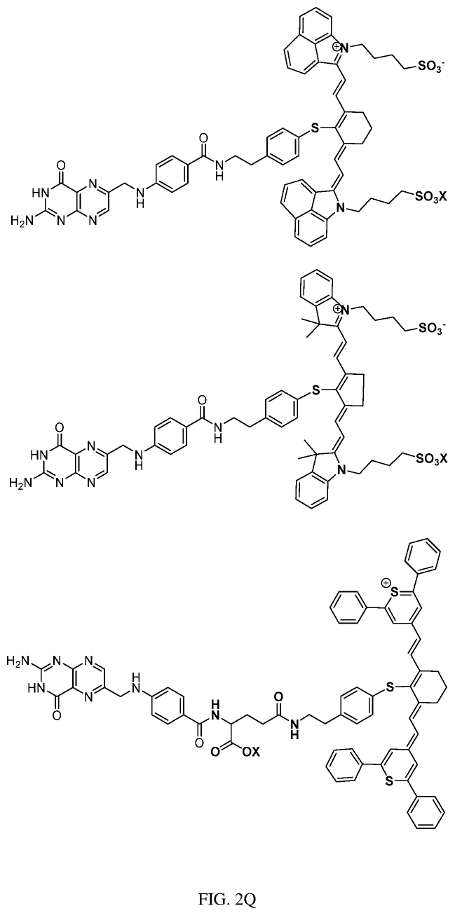

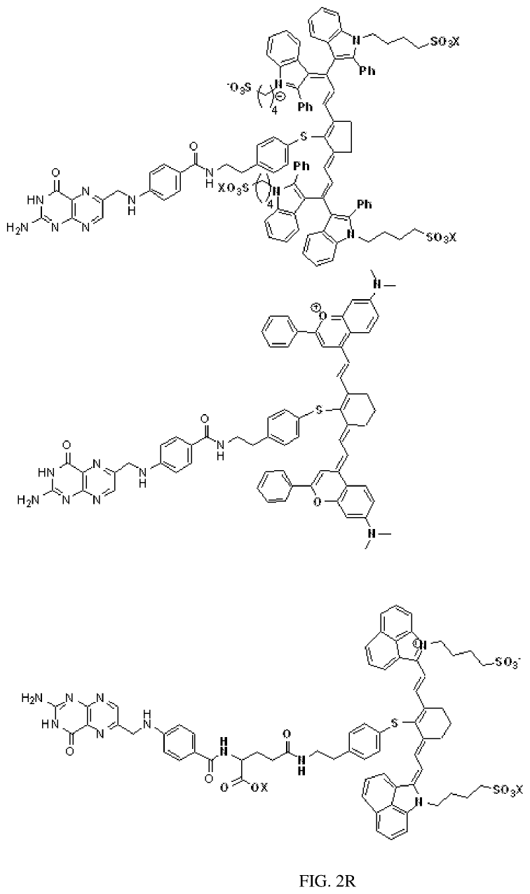

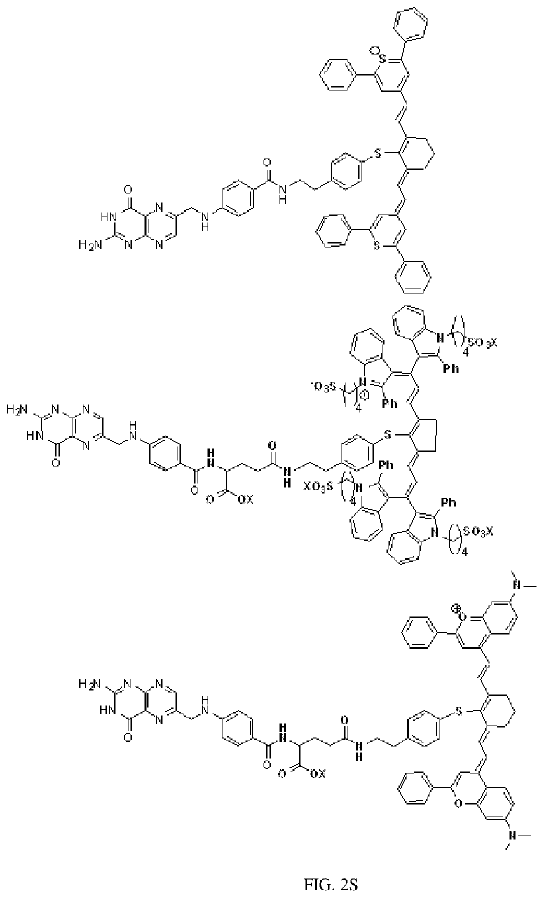

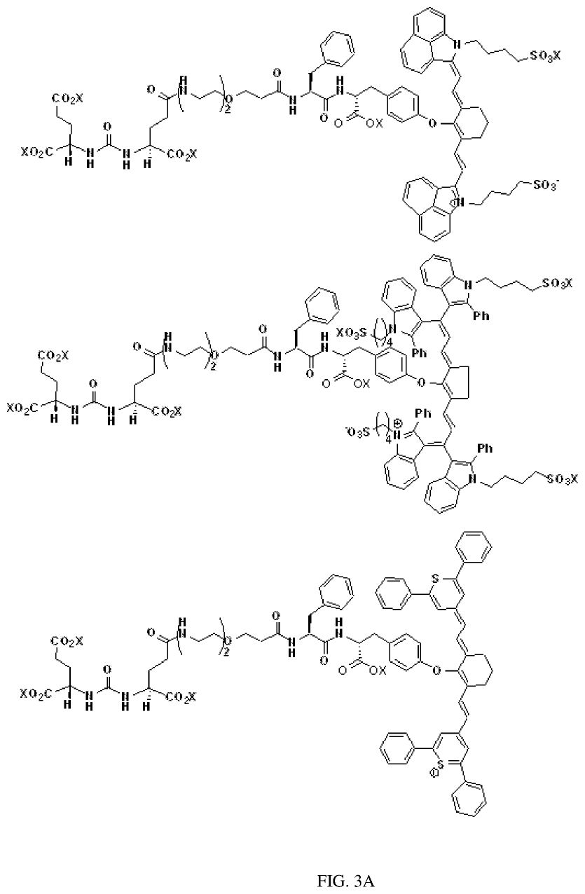

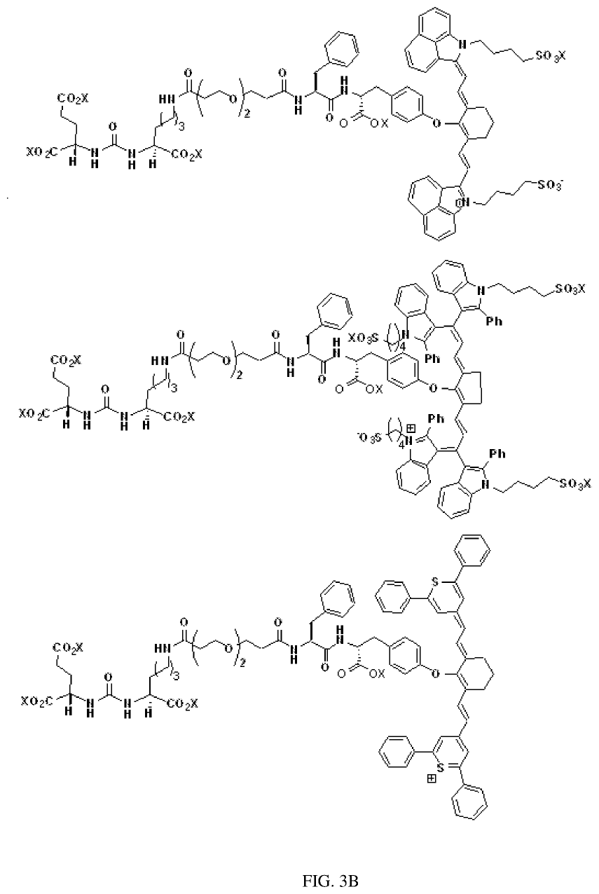

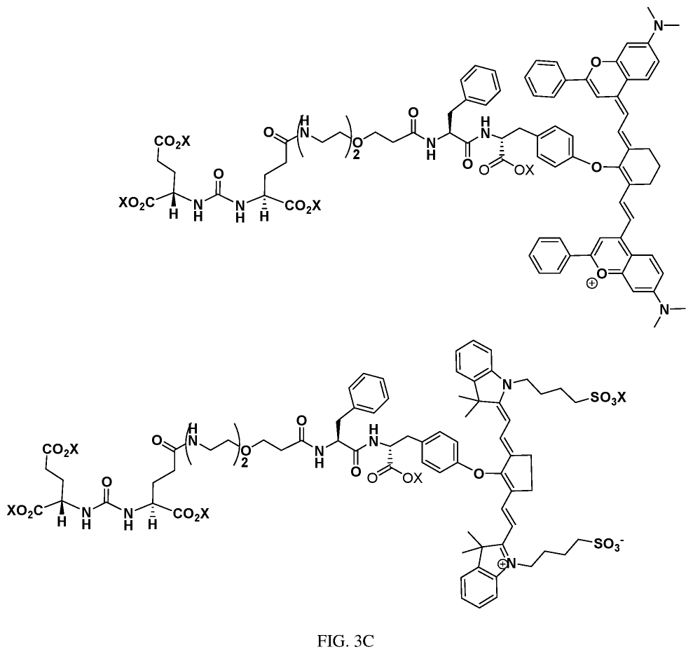

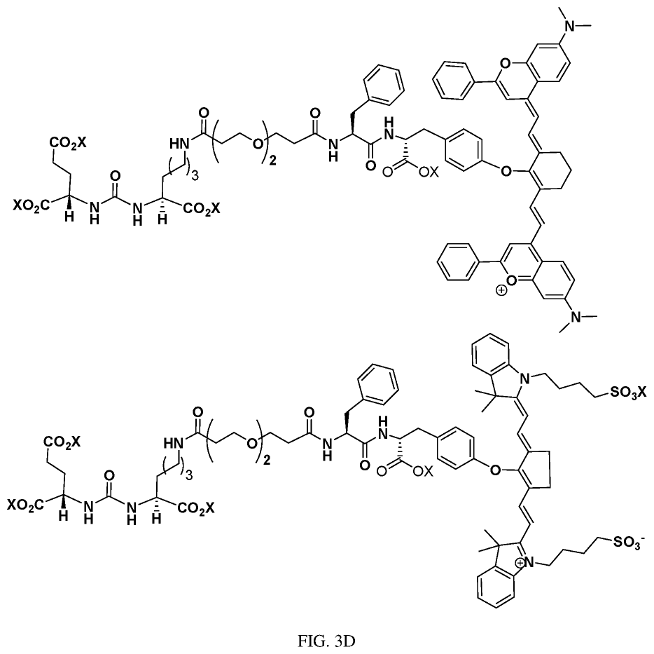

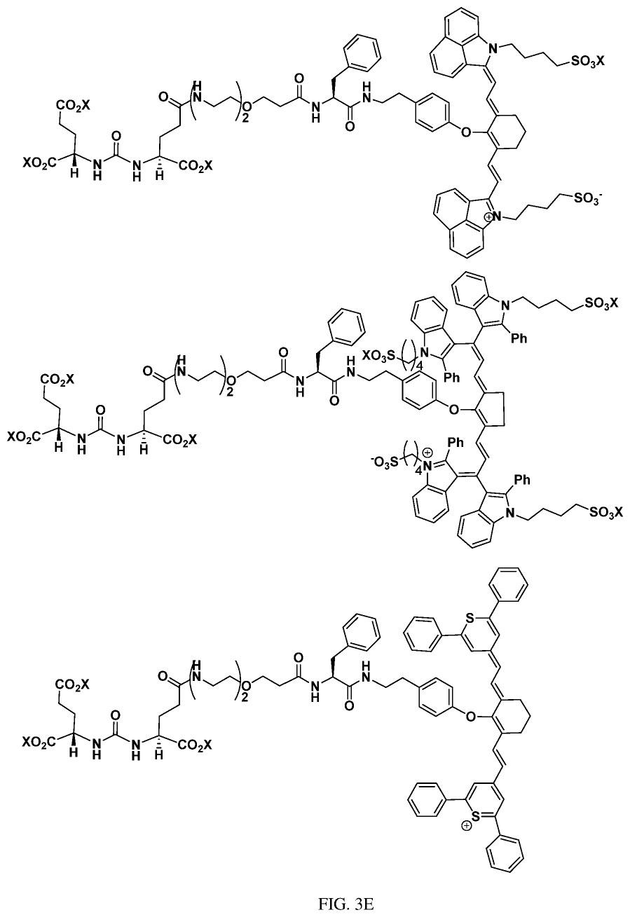

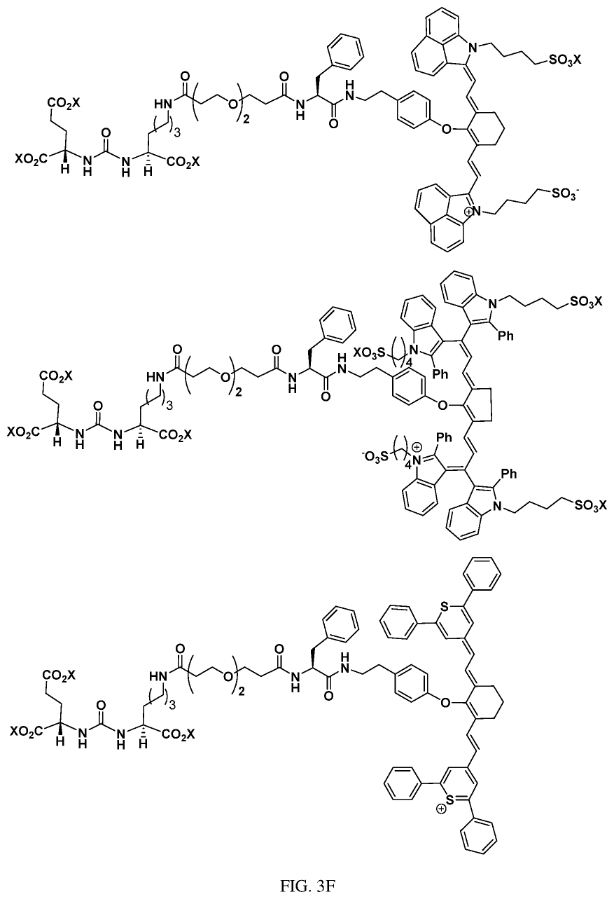

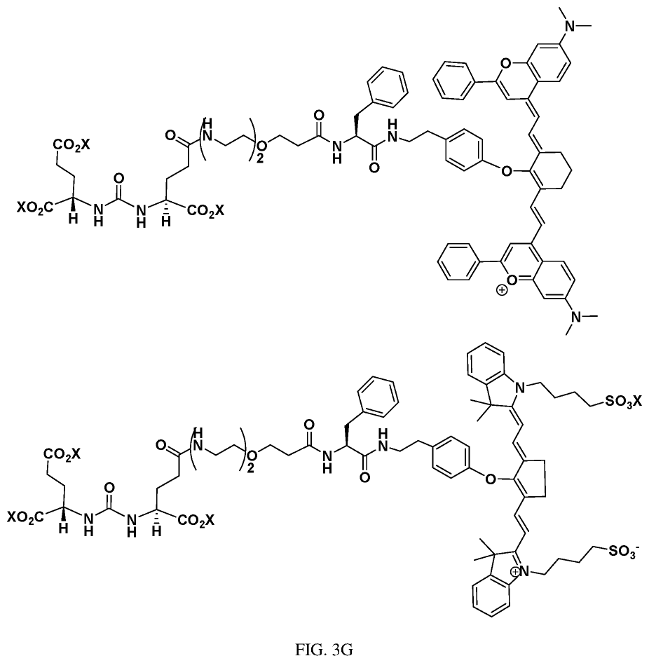

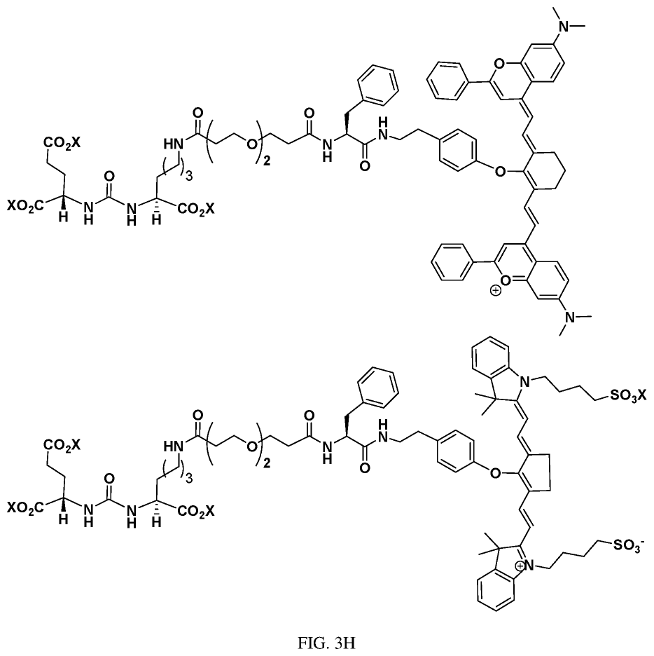

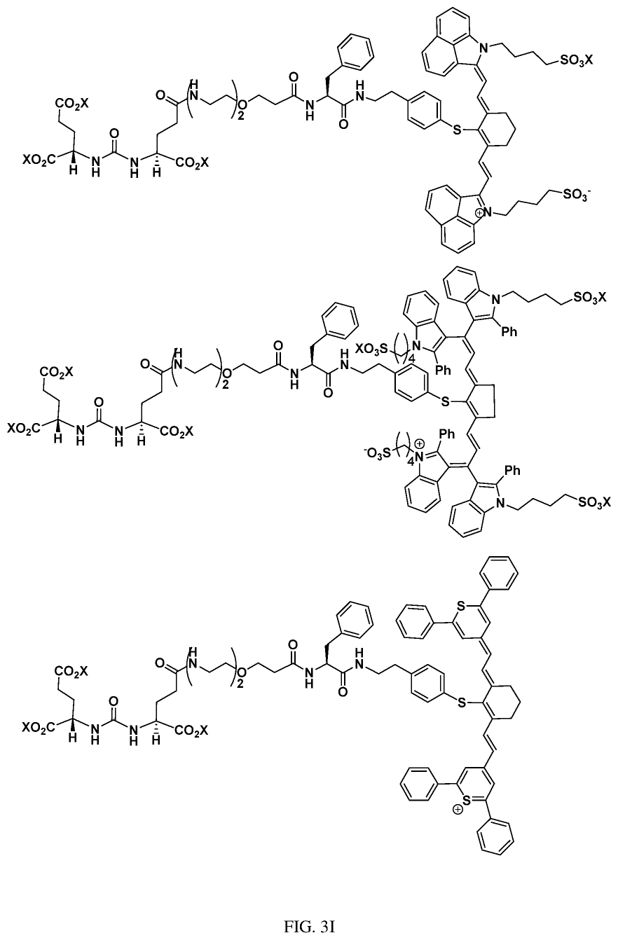

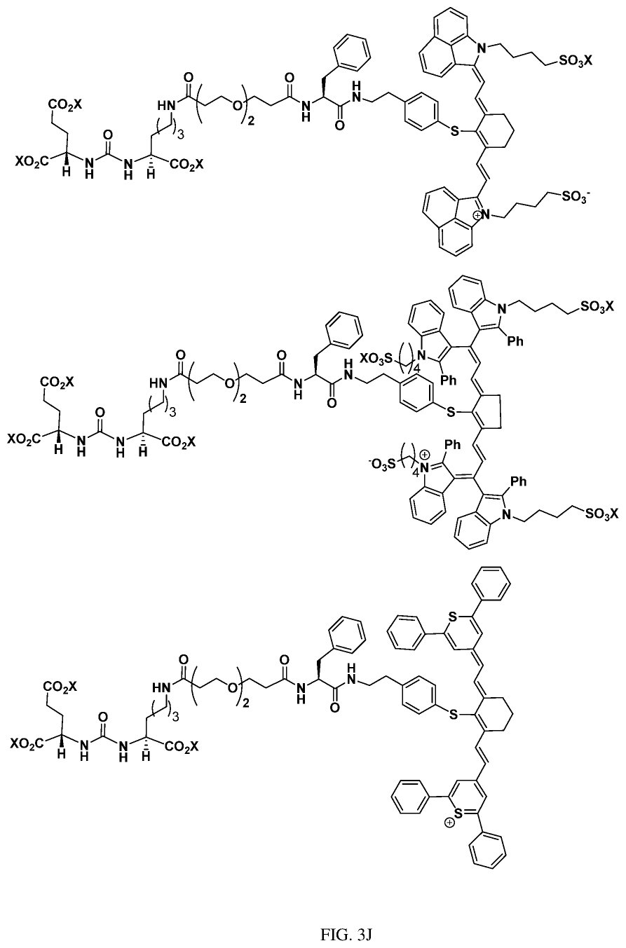

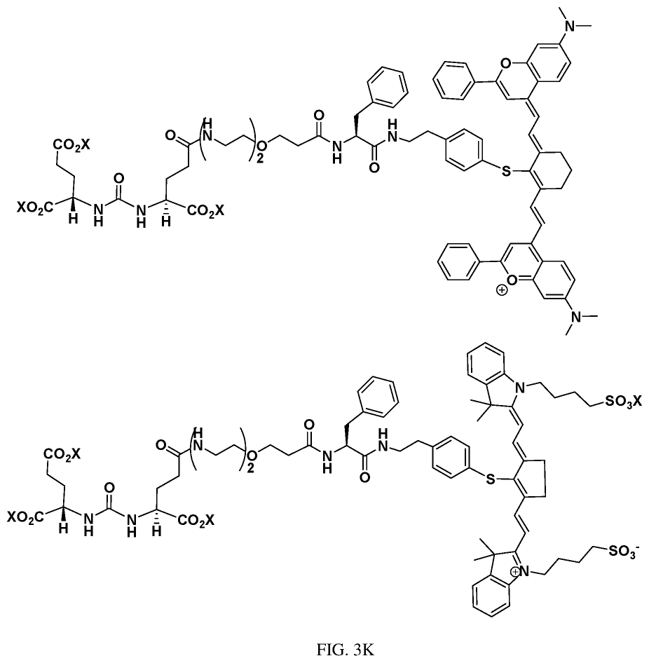

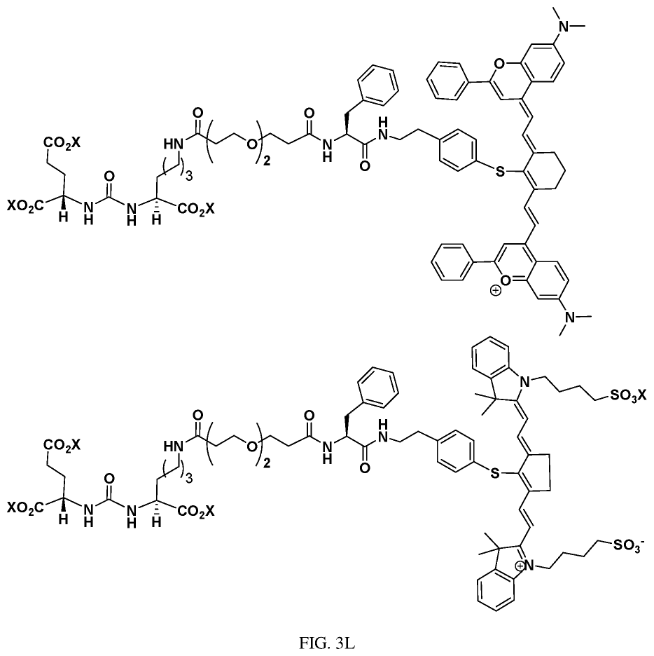

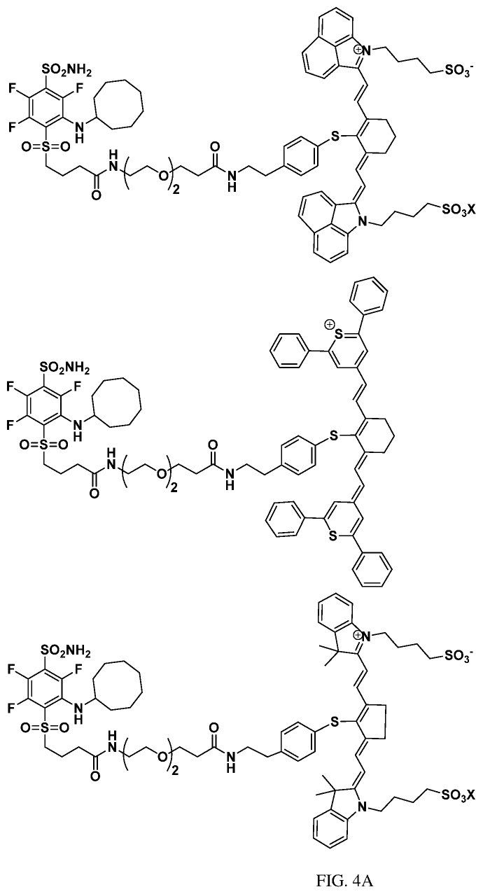

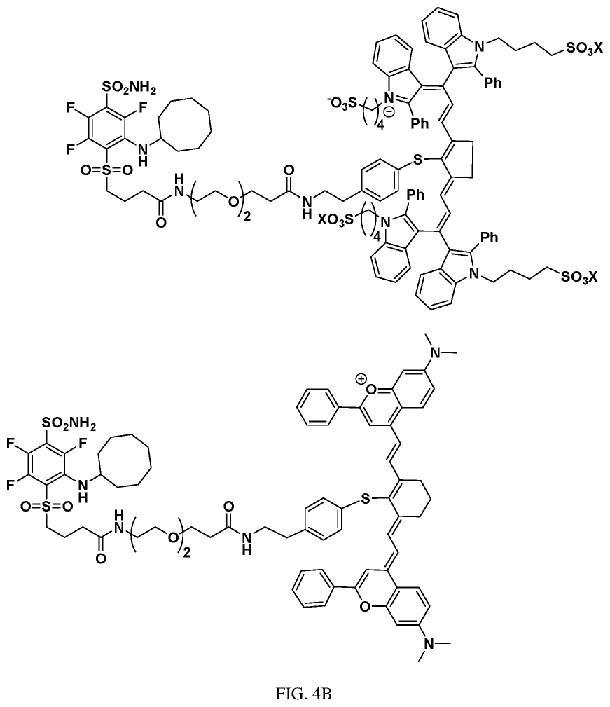

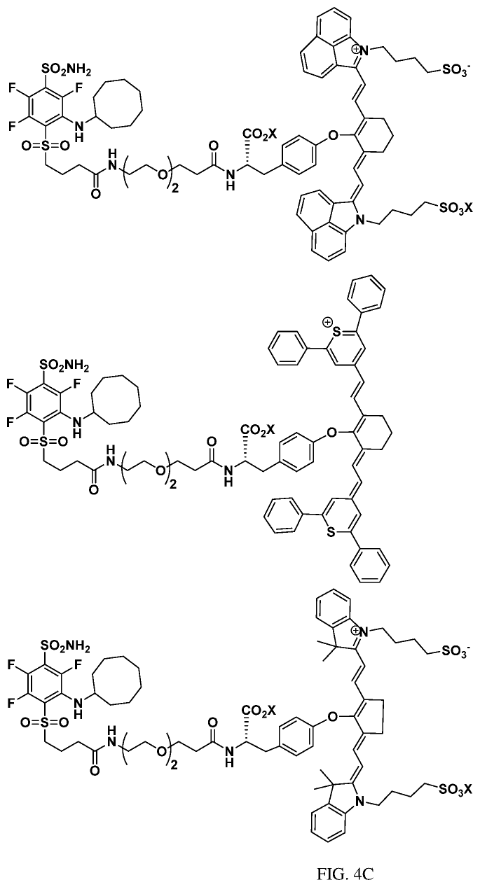

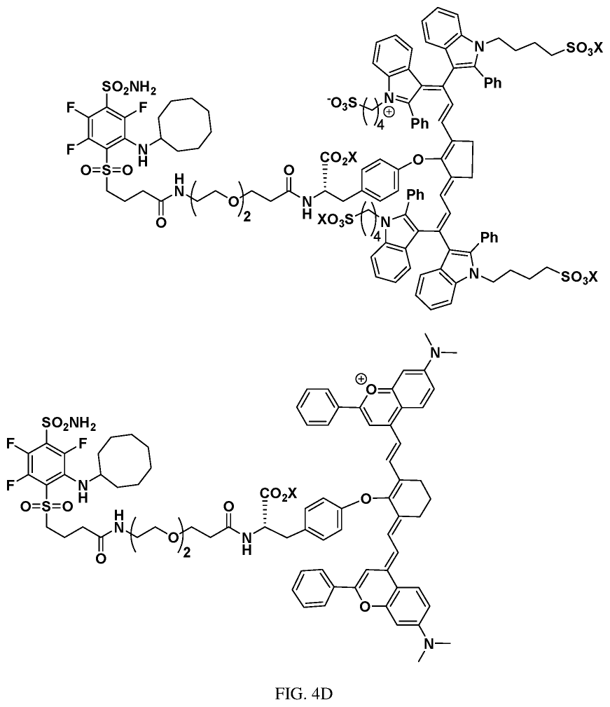

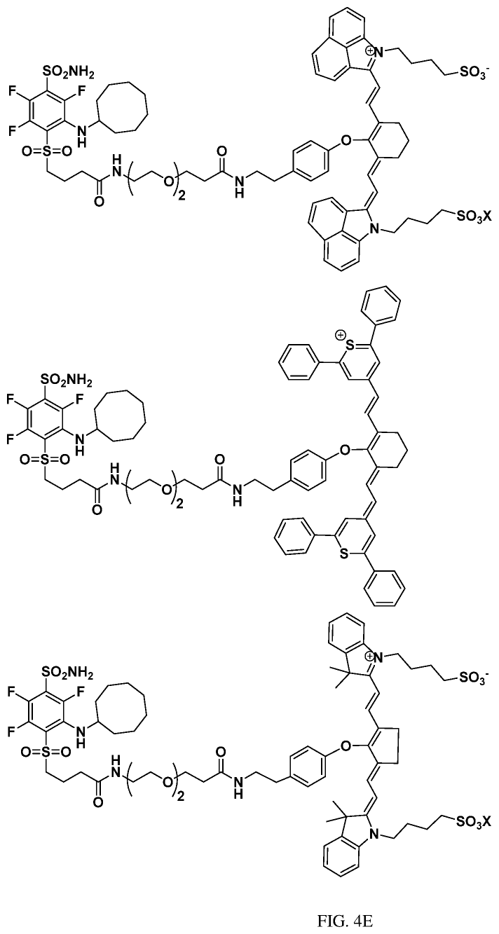

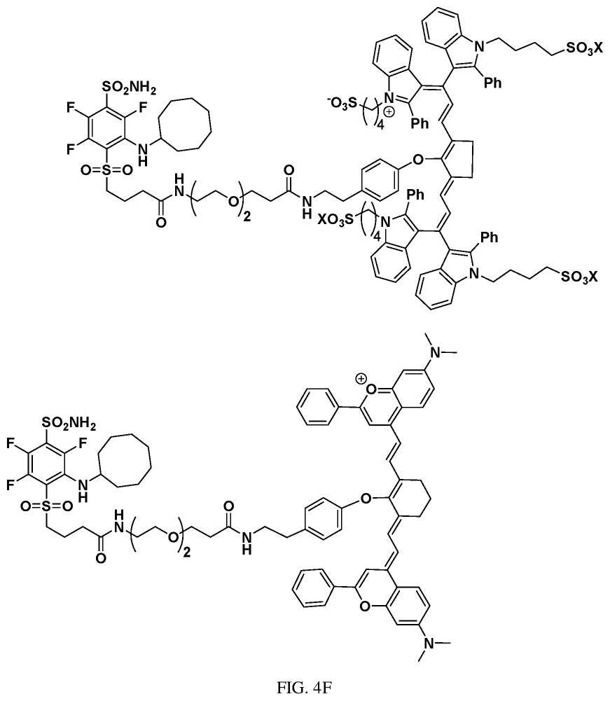

The targeting moiety of the compound targets a receptor, antigen, or protein. In some aspect, the tumor targeting ligand can be used to detect target tissue, target cells, or CTCs that have folate receptor that binds to folic acid, a folic acid analog, or another folate receptor-binding molecule. Exemplary several folate receptor-targeted NIR-II imaging agents are shown in FIGS. 2A-2S. In other aspects, the targeting moiety can be used to detect target tissue, target cells, or CTCs that have prostate-specific membrane antigen (PSMA) or another prostate cancer-specific binding molecule. Exemplary PSMA-targeted NIR-II imaging agents are shown in FIGS. 3A-3L. In other aspects, the targeting moiety can be used to detect target tissue, target cells, or CTCs that have glutamate carboxypeptidase II, carbonic anhydrase IX (CA IX) (FIGS. 4A-4F), fibroblast activation protein alpha, glucose transporter 1, cholecystokinin-2, or other receptors, antigens, and/or antibodies commonly found in cancer cells.

Because not all cancers express the same receptor, antigen, and/or antibody, it is contemplated that several compounds that target different receptors, antigens, and/or antibodies can be used in series or in combination.

In one aspect of the method, a bodily fluid from the subject is contracted with the compound. The bodily fluid includes, but is not limited to, urine, nasal secretions, nasal washes, bronchial lavages, bronchial washes, spinal fluid, sputum, gastric secretions, reproductive tract secretions (e.g., seminal fluid), lymph fluid, mucus, and blood. In some aspects, the compound is in contact with the bodily fluid for at least 30 minutes, alternatively at least 1 hour, alternatively at least 2 hours, alternatively at least 3 hours.

In a further aspect of the methods provided, the cancer cells are of a tumor. In some aspects, the volume of the tumor is at least 1000 mm³. In some aspects, the volume of the tumor is less than 1000 mm³. In some aspects, the volume of the tumor is less than 950 mm³. In some aspects, the volume of the tumor is less than 900 mm³. In some aspects, the volume of the tumor is less than 850 mm³. In some aspects, the volume of the tumor is less than 800 mm³. In some aspects, the volume of the tumor is less than 750 mm³. In some aspects, the volume of the tumor is less than 700 mm³. In some aspects, the volume of the tumor is less than 650 mm³. In some aspects, the volume of the tumor is less than 600 mm³. In some aspects, the volume of the tumor is less than 550 mm³. In some aspects, the volume of the tumor is less than 500 mm³. In some aspects, the volume of the tumor is less than 450 mm³. In some aspects, the volume of the tumor is less than 400 mm³. In some aspects, the volume of the tumor is less than 350 mm³. In some aspects, the volume of the tumor is less than 300 mm³. In some aspects, the volume of the tumor is less than 250 mm³. In some aspects, the volume of the tumor is less than 200 mm³. In some aspects, the volume of the tumor is less than 150 mm³. In some aspects, the volume of the tumor is less than 100 mm³. In one aspect, the volume of the tumor is at least 75 mm³. In another aspect, the volume of the tumor is less than 75 mm³. In another aspect, the volume of the tumor is less than 70 mm³. In another aspect, the volume of the tumor is less than 65 mm³. In another aspect, the volume of the tumor is less than 60 mm³. In another aspect, the volume of the tumor is less than 55 mm³. In one aspect, the volume of the tumor is at least 50 mm³. In other aspects, the tumor is less than 50 mm³. In another aspect, the volume of the tumor is less than 45 mm³. In other aspects, the volume of the tumor is less than 40 mm³. In another aspect, the volume of the tumor is less than 35 mm³. In still another aspect, the volume of the tumor is less than 30 mm³. In another aspect, the volume of the tumor is less than 25 mm³. In still another aspect, the volume of the tumor is less than 20 mm³. In another aspect, the volume of the tumor is less than 15 mm³. In still another aspect, the volume of the tumor is less than 10 mm³. In still another aspect, the volume of the tumor is less than 12 mm³. In still another aspect, the volume of the tumor is less than 9 mm³. In still another aspect, the volume of the tumor is less than 8 mm³. In still another aspect, the volume of the tumor is less than 7 mm³. In still another aspect, the volume of the tumor is less than 6 mm³. In still another aspect, the volume of the tumor is less than 5 mm³.

In one aspect, the tumor has a length of at least 5 mm prior to surgical recision using a NIR-II dye compound of the present invention. In one aspect, these methods detect tumors less than 5 mm. In other aspects, the methods herein detect tumors less than 4 mm. In some aspects, the methods herein detect tumors less than 3 mm. In another aspect, the tumor has a length of at least 6 mm. In still another aspect, the tumor has a length of at least 7 mm. In yet another aspect, the tumor has a length of at least 8 mm. In another aspect, the tumor has a length of at least 9 mm. In still another aspect, the tumor has a length of at least 10 mm. In yet another aspect, the tumor has a length of at least 11 mm. In a further aspect, the tumor has a length of at least 12 mm. In still a further aspect, the tumor has a length of at least 13 mm. In still a further aspect, the tumor has a length of at least 14 mm. In another aspect, the tumor has a length of at least 15 mm. In yet another aspect, the tumor has a length of at least 16 mm. In still another aspect, the tumor has a length of at least 17 mm. In a further aspect, the tumor has a length of at least 18 mm. In yet a further aspect, the tumor has a length of at least 19 mm. In still a further aspect, the tumor has a length of at least In another aspect, the tumor has a length of at least 21 mm. In still another aspect, the tumor has a length of at least 22 mm. In yet another aspect, the tumor has a length of at least 23 mm. In a further aspect, the tumor has a length of at least 24 mm. In still a further aspect, the tumor has a length of at least 25 mm. In yet a further aspect, the tumor has a length of at least 30 mm.

In some aspects, the present disclosure relates to NIR-II dye compounds and methods for their therapeutic and diagnostic use. More specifically, this disclosure provides compounds and methods for diagnosing and treating diseases associated with cells expressing receptors, antigens, or antibodies targeted by the tumor targeting ligand, such as cancer and related diseases. The disclosure further describes methods and compositions for making and using the compounds, methods incorporating the compounds, and kits incorporating the compounds.

In one illustrative aspect, the linker L may be a releasable or non-releasable linker. In one aspect, the linker L is at least about 6 atoms in length. In one variation, the linker L is at least about 10 atoms in length. In one variation, the linker L is at least about 14 atoms in length. In another variation, the linker L is between about 6 and about 22, between about 6 and about 24, or between about 6 and about 20 atoms in length. In another variation, the linker L is between about 14 and about 31, between about 14 and about 24, or between about 14 and about 20 atoms in length.

In another aspect, pharmaceutical compositions are described herein, where the pharmaceutical composition includes the compounds described herein in amounts effective to treat diseases and disease states, diagnose diseases or disease states, and/or image tissues and/or cells that are associated with pathogenic populations of cells expressing or over expressing a target receptor, antigen, or antibody. Illustratively, the pharmaceutical compositions also include one or more carriers, diluents, and/or excipients.

In another aspect, methods for treating diseases and disease states, diagnosing diseases or disease states, and/or imaging tissues and/or cells that are associated with pathogenic populations of cells expressing or over expressing the targeted receptors, antigens, or antibodies are described herein. Such methods include the step of administering the compounds described herein, and/or pharmaceutical compositions containing the compounds described herein, in amounts effective to treat diseases and disease states, diagnose diseases or disease states, and/or image tissues and/or cells that are associated with pathogenic populations of cells expressing or over expressing the target receptor, antigen, or antibody.

The identification of small tumors will lead to a more accurate and more effective resection of the primary tumor to produce negative margins, as well as accurate identification and removal of the lymph nodes harboring metastatic cancer cells and identification of satellite disease. Each of these advantages positively correlates with a better clinical outcome for the patient being treated.

The compounds can be used with fluorescence-mediated molecular tomographic imaging systems, such as those designed to detect near-infrared fluorescence activation in deep tissues. The compounds provide molecular and tissue specificity, yield high fluorescence contrast, brighter fluorescence signal, and reduce background autofluorescence, allowing for improved early detection and molecular target assessment of diseased tissue in vivo (e.g., cancers). The compounds can be used for deep tissue three-dimensional imaging, targeted surgery, and methods for quantifying the amount of a target cell type in a biological sample.

In some aspects, the present technology may be used in a method for detecting CTCs to provide real-time monitoring, screening, and management of a subject having a disease.

In some aspects, the present technology may be used in a method of detecting the presence of CTCs to determine the likelihood of the recurrence or remission of a disease in a subject. In some aspects of the methods, after 30 minutes, to allow for clearance of unbound compounds, blood is drawn from the subject, and multiphoton intravital microscopy is used to detect CTCs. To achieve a quantitative analysis of these CTCs in larger, faster flowing vessels, fluorescence scanning is reduced to a single dimension along a transect perpendicular to the vessel. This modification allows an increase in scan rate from 2 to 500 frames per second.

In an exemplary embodiment, CTCs originating from a primary solid tumor are quantitated in vivo before the metastatic disease is detectable by microscopic examination of necropsied tissues.

In an exemplary embodiment, CTCs from a human or animal subject with cancer are detected in whole blood. The human or animal subject is treated with a compound that comprises a targeting moiety and a fluorescence-imaging agent, wherein the targeting moiety targets a receptor, antigen, or antibody and the collected blood samples are examined by flow cytometry. To confirm the labeled CTCs are malignant, the peripheral blood samples from the subjects are labeled with a monoclonal antibody and an appropriate secondary antibody conjugated to a fluorescence-imaging agent.

The NIR-II dye compounds of the present invention produce a tumor-to-background signal ratio that is higher than the tumor-to-background signal ratio compared to a similar non-NIR dye or non-targeted NIR dye compound.

It will be apparent to those skilled in the art that various changes may be made in the disclosure without departing from the spirit and scope thereof. Therefore, the disclosure encompasses embodiments in addition to those specifically disclosed in the specification and as indicated in the appended claims.

Further aspects and embodiments of the present technology are described in the following paragraphs.

A compound of the present disclosure, or pharmaceutically acceptable salt of the compound, wherein the compound has the formula B-L-X, wherein B is a tumor-targeted ligand, L is a linker, and X is a NIR-II dye.

A compound of the present disclosure, wherein the pharmaceutically acceptable salt selected from the group consisting of sodium, potassium, ammonium, calcium, magnesium, lithium, cholinate, lysinium, and hydrogen salt.

A compound of the present disclosure, wherein B targets a biomarker, receptor, protein, antigen, or enzyme.

A compound of the present disclosure, wherein B targets a folate receptor, Glutamate carboxypeptidase II, prostate-specific membrane antigen, carbonic anhydrase IX (CA IX), Fibroblast activation protein alpha, Glucose transporter 1, or cholecystokinin-2.

A compound of the present disclosure, wherein L is a hydrophilic spacer, an amino acid, a peptide or a derivative thereof, a polyether, a sulfonic acid or a derivative thereof, or a glycan or a derivative thereof.

A compound of the present disclosure, wherein X is a NIR-II dye selected from the group consisting of:

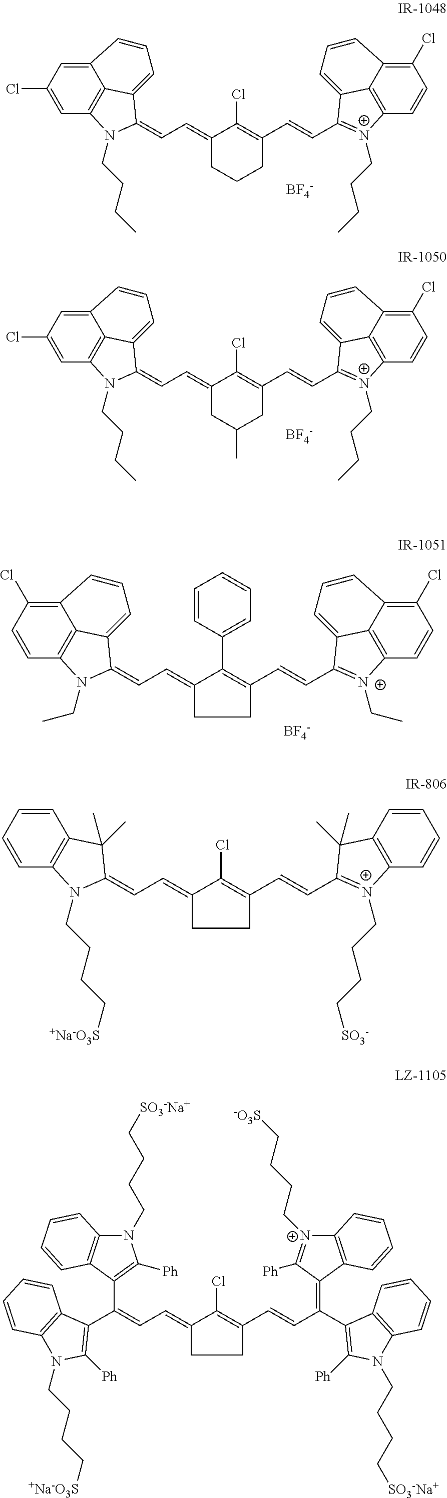

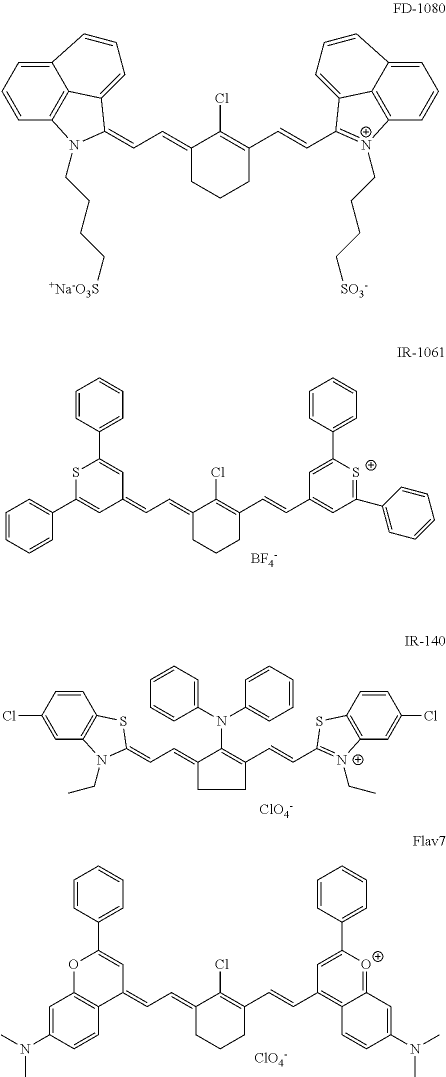

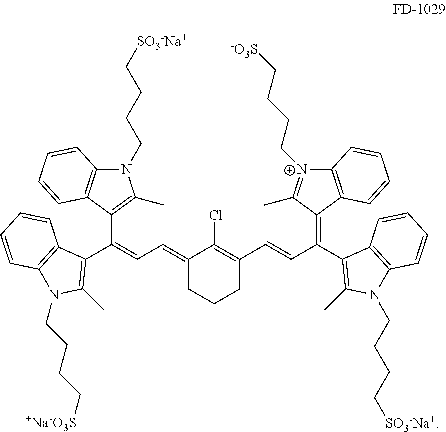

A composition comprising a compound of the present disclosure and a pharmaceutically acceptable carrier, excipient, or diluent.

A compound of the present disclosure, wherein the composition comprises a pharmaceutically or therapeutically acceptable amount of the compound.

A kit comprising a compound of the present disclosure.

A method of identifying a target cell, target tissue, or CTCs in a biological sample, the method comprising:
(a) contacting the biological sample with a compound of the present disclosure for a time and under conditions sufficient for binding of the compound to the target cell, target tissue, or CTCs;
(b) optically detecting the presence or absence of the compound in the biological sample;
wherein the presence of the compound in detecting step (b) indicates that the target cell, target tissue, or CTCs is present in the biological sample.

A method of performing image-guided surgery on a subject, the method comprising:
(a) administering a compound of the present disclosure to the subject for a time and under conditions sufficient for the compound to accumulate at a surgical site of the subject;
(b) illuminating the surgical site to visualize the compound using near infrared light; and
(c) performing surgical resection of tissue that fluoresces upon excitation with the infrared light.

A method of diagnosing a disease in a subject, the method comprising:
(a) administering a compound of the present disclosure to the subject for a time and under conditions sufficient for binding of the compound to a target cell in a tissue of the subject;
(b) illuminating the tissue to visualize the compound using infrared light;
(c) measuring a fluorescent signal from the compound upon excitation with the infrared light;
(d) comparing the fluorescent signal measured in (c) with at least one control data set, wherein the at least one control data set comprises a fluorescent signal from the compound of the present disclosure contacted with a biological sample that does not comprise the target cell; and
(e) diagnosing the subject with the disease, wherein the comparison in (d) indicates the presence of the disease.

A method of the present disclosure, wherein the disease is selected from the group consisting of cancer, cardiovascular disease, neurodegenerative disease, immunologic disease, autoimmune disease, respiratory disease, metabolic disease, inherited disease, infectious disease, bone disease, and environmental disease.

A method of optical or diagnostic imaging of a biological tissue that expresses a folate receptor, Glutamate carboxypeptidase II, prostate-specific membrane antigen, carbonic anhydrase IX (CA IX), Fibroblast activation protein alpha, Glucose transporter 1, or cholecystokinin-2, the method comprising:
(a) contacting the biological tissue of a subject with a compound of the present disclosure;
(b) allowing time for the compound to distribute within the biological tissue;
(c) illuminating the biological tissue with an excitation wavelength absorbable by the compound; and
(d) detecting an optical signal emitted by the compound.

A method of the present disclosure, wherein the optical signal emitted by the compound is used to construct an image.

A method of the present disclosure, wherein diagnostic imaging is fluorescence-guided surgery or image-guided surgery.

A method of the present disclosure, wherein the biological tissue is selected from the group consisting of diseased tissue, abnormal tissue, tumor lesions, and lymph nodes with metastatic tumor cells.

A method of the present disclosure wherein the diagnostic imaging further comprises:
(e) diagnosing the subject with cancer.

A method of imaging cancer cells or CTCs that express a folate, PSMA, or CA-IX receptor in a subject, the method comprising:
(a) administering to the subject a compound of the present disclosure for a time and under conditions sufficient for binding of the compound to the cancer cells; and
(b) fluorescence-imaging of tissue of the subject, wherein the tissue comprises the compound bound to the cancer cells.

A method of the present disclosure, wherein cancer cells or CTCs are selected from the group consisting of prostate cancer cells, cervical cancer cells, ovarian cancer cells, endometrial cancer cells, brain cancer cells, breast cancer cells, leukemia cells, kidney cancer cells, head and neck cancer cells, esophageal cancer cells, liver cancer cells, and lung cancer cells.

A method of the present disclosure, wherein the methods employ bioluminescence resonance energy transfer (BRET) or two-step fluorescence resonance energy transfer (FRET).

A method of the present disclosure, wherein the compound is used in a multifunctional imaging technique.

A method for detecting CTCs to provide real-time monitoring, screening, and management of subject having a disease, wherein the method comprises the detection of CTCs using a compound of the present disclosure.

A method of detecting the presence of CTCs to determine the likelihood of the recurrence or remission of a disease in a subject, wherein the method comprises the detection of CTCs using a compound of the present disclosure.

A method of detecting the presence of CTCs to determine the likelihood of response to surgical treatment, chemotherapy, immunotherapy, radiotherapy, hormonal therapy, wherein the method comprises the detection of CTCs using a compound of the present disclosure.

A method of the present disclosure, wherein the subject is a mammal.

A method of the present disclosure, wherein the subject is a human.

A method of the present disclosure, wherein the subject has a disease.

A method of the present disclosure, wherein the subject has cancer.

A method of the present disclosure, wherein the subject has early-stage cancer or metastatic cancer.

A method of the present disclosure, wherein the subject has cancer and the cancer is selected from the group consisting of pancreatic, gastrointestinal, stomach, colon, ovarian, cervical, prostate, glioma, carcinoid, or thyroid, lung cancer, bladder cancer, liver cancer, kidney cancer, sarcoma, breast cancer, brain cancer, testicular cancer, and melanoma.

A method of the present disclosure, wherein the CTCs are from a cancerous tumor, specifically a primary tumor.

A method of the present disclosure, wherein the bodily fluid, is selected from the group consisting of urine, nasal secretions, nasal washes, bronchial lavages, bronchial washes, spinal fluid, sputum, gastric secretions, reproductive tract secretions, lymph fluid, mucus, and blood.

A method of the present disclosure, wherein the compound is in contact with the bodily fluid for at least 30 minutes, alternatively at least 1 hour, alternatively at least 2 hours, alternatively at least 3 hours.

A method of the present disclosure wherein the method is performed in vitro, in vivo, or ex vivo.

A method of the present disclosure, wherein the method is performed in vivo and target cells, target tissue, or CTCs are detected using two-photon microscopy, epifluorescence microscopic, or an innovative wearable.

A method of the present disclosure, wherein the innovative wearable is a smartwatch, a wristband, earpiece, wearable microscope, or bicep band.

A method of the present disclosure, wherein the innovative wearable is a smartwatch, wherein the smartwatch employs sensors and algorithms for detecting and quantifying a subject's CTC levels.

A method of the present disclosure, wherein the innovative wearable is a wearable microscope.

A method of the present disclosure, wherein the wearable microscope employs lasers to generate a fluorescent image.

A method of the present disclosure, wherein if an abnormal CTC level is detected, the subject is notified of the potential abnormality.

A method of the present disclosure, wherein the CTC levels are continuously monitored.

A method of the present disclosure, wherein the method is used to track and analyze the distribution and the phenotype of cancer cells.

A method of the present disclosure, wherein the detected CTCs are further isolated and/or enriched using ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods.

A method for detecting CTCs to provide real-time monitoring, screening, and management of subject having a disease, wherein the method comprises the detection of CTCs using a compound of the present disclosure and the real-time monitoring, screening, and management is tracked through a software platform or is delivered to an innovative wearable

A method of the present disclosure, wherein the method comprises contacting a bodily fluid of the subject with the compound for a time and under conditions that allow for binding of the compound to at least one CTC, illuminating the CTCs with an excitation light of a wavelength that is absorbed by the compound emitted by the innovative wearable, and detecting the optical signal emitted by the compound.

A compound of the present disclosure, a composition of the present disclosure, a kit of the present disclosure, or a method of the present disclosure, wherein the NIR-II dye has an excitation and emission spectra in the second region near infrared.

A compound of the present disclosure, a composition of the present disclosure, a kit of the present disclosure, or a method of the present disclosure, wherein the NIR-II dye has an absorption and emission maxima between about 1000 nm and 1700 nm.

### REFERENCES

1. De Grand, A. M., Frangioni, J. V. (2003) An operational near-infrared fluorescence imaging system prototype for large animal surgery. Technol. Cancer. Res. Treat. 2, 553-562.
2. Griffiths, C. T. (1975) Surgical resection of tumor bulk in the primary treatment of ovarian carcinoma. Natl Cancer Inst Monogr. 42, 101-104.
3. Bristow, R. E., Tomacruz, R. S., Armstrong, D. K., Trimble, E. L., Montz, F. J. (2002) Survival effect of maximal cytoreductive surgery for advanced ovarian carcinoma during the platinum era: A meta-analysis. J. Clin. Oncol. 20, 1248-1259.
4. Karakiewicz, P. I., Eastham, J. A., Graefen, M., Cagiannos, I., Stricker, P. D., Klein, E., Cangiano, T., Schroder, F. H., Scardino, P. T., Kattan, M. W. (2005) Prognostic impact of positive surgical margins in surgically treated prostate cancer: multi-institutional assessment of 5831 patients. Urololgy. 66, 1245-1250.
5. Kapp, K. S., Kapp, D. S., Poschauko, J. , Stucklschweiger, G. F., Hackl, A., Pickel, H., Petru, E., Winter, R. (1999) The prognostic significance of peritoneal seeding and size of postsurgical residual in patients with stage III epithelial ovarian cancer treated with surgery, chemotherapy and radiotherapy. Genecol. Oncol. 74, 400-407.
6. Kitai, T., Inomoto, T., Miwa, M., Shikayama, T. (2005) Fluorescence navigation with indocyanine green for detecting sentinel lymph nodes in breast cancer. Breast Cancer. 12, 211-215.
7. Tagaya, N., Yamazaki, R., Nakagawa, A., Abe, A., Hamada, K., Kubota, K., Oyama, T. (2008) Intraoperative identification of sentinel lymph nodes by near-infrared fluorescence imaging in patients with break cancer. Am. J. Surg. 195, 850-853.
8. Fujiwara, M., Mizukami, T., Suzuki, A., Fukamizu, H. (2009). Sentinel lymph node detection in skin cancer patients using real-time fluorescence navigation with indocyanine green: preliminary experience. J. Plast. Reconstr. Aesthet. Surg. 62, E373-E378.
9. Ogasawara, Y., Ikeda, H., Takahashi, M., Kawasaki, K., Doihara, H. (2008) Evaluation of breast lympathic pathways with indocyanine green fluorescence imaging in patients with breast cancer. World J Surg, 32, 1924-1929.
10. Recent Progress in NIR-II Contrast Agent for Biological Imaging. Cao J, Zhu B, Zheng K, He S, Meng L, Song J, Yang H. Front Bioeng Biotechnol. 2020 Jan. 30; 7:487.
11. Weissleder, R., Tung, C. H., Mahmood, U., Bogdanov, A. Jr. (1999) In vivo imaging of tumors with protease-activated near-infrared fluorescent probes. Nat. Biotechnol. 17, 375-387.
12. Urano, Y., Asanuma, D., Hama, Y. (2009) Selective molecular imaging of viable cacer cells with pH-actiavatable fluorescence probes. Nat. Med. 15, 104-109.
13. Mahmood, U., Weissleder, R. (2003) Near-infrared optical imaging of proteases in cancer. Mol. Cancer Ther. 2, 489-496.
14. Hama, Y., Urano, Y., Koyama, Y., Gunn, J. R., Choyke, P. L., Kobayashi, H. (2007) A self-quenched galactosamine-serum albumin-rhodamineX conjugate a ‘smart’ fluorescent molecular imaging probe synthesized with clinically applicable material for detecting peritoneal ovarian cancer metastases. Clin. Cancer Res. 13, 6335-6343.
15. Ogawa, M., Kosaka, N., Choyke, P. L., Kobayashi, H. (2009) In vivo molecular imaging of cancer with a quenching near-infrared fluorescent probe using conjugates of monoclonal antibodies and indocyanine green. Cancer Res. 69. 1268-1272.
16. Bremer, C., Bredow, S., Mahmood, U., Weissleder, R., Tung, C. H. (2001) Optical imaging of matrix metalloproteinase-2 activity in tumors: feasibility study in a mouse model. Radiology. 221, 523-529.
17. Mahmood, U., Tung, C. H., Bogdanov, A. Jr., Weissleder, R. (1999) Near-infrared optical imaging of protease activity for tumor detection. Radiology. 213, 866-870.
18. Kularatne, S. A., Wang, K., Santhapuram, H. K. R., Low, P. S. (2009) Prostate-specific membrane antigen targeted imaging and therapy of prostate cancer using a PSMA inhibitor as a homing ligand. Molecular Pharmaceutics. 6, 780-789.
19. Kularatne, S. A., Zhou, Z., Yang, J., Post, C. B., Low, P. S. (2009) Design, synthesis and preclinical evaluation of prostate-specific membrane antigen targeted 99mTc-radioimaging agents. Molecular Pharmaceutics. 6, 790-800.
20. Kularatne, S. A., Thomas, M, Myers C. H., Gagare, P, Kanduluru, A. K., Crian, C. J., Cichocki, B. N. Evaluation of Novel Prostate-Specific Membrane Antigen-Targeted Near-Infrared Imaging Agent for Fluorescence-Guided Surgery of Prostate Cancer. Clin Cancer Res. 25, 177-187.
21. Mahalingam, S. M., Kularatne, S. A., Myers, C. H., Gagare, P, Norshi, M, Liu, X, Singhal, S, Low, P. S. Evaluation of Novel Tumor-Targeted Near-Infrared Probe for Fluorescence-Guided Surgery of Cancer. J Med Chem., 61, 9637-9646.
22. Low, P. S., Kularatne, S. A. Folate-targeted therapeutic and imaging agents for cancer. Curr Opin Chem Biol., 13, 256-62.

While the present invention has been described with reference to certain aspects, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all aspects falling within the scope of the appended claims.

1. A compound, or pharmaceutically acceptable salt of the compound, wherein the compound has the formula B-L-X,
wherein B is a tumor targeted ligand which targets a folate receptor, Glutamate carboxypeptidase II, prostate-specific membrane antigen (PSMA), carbonic anhydrase IX (CA IX), Fibroblast activation protein alpha, Glucose transporter 1, or cholecystokinin,
L is selected from the group consisting of a hydrophilic spacer, an amino acid, a peptidic or derivatives thereof, a polyether, a sulfonic acid or derivatives thereof, a glycans or derivatives thereof, and combinations thereof,
and X is a NIR-II dye selected from the group consisting of:

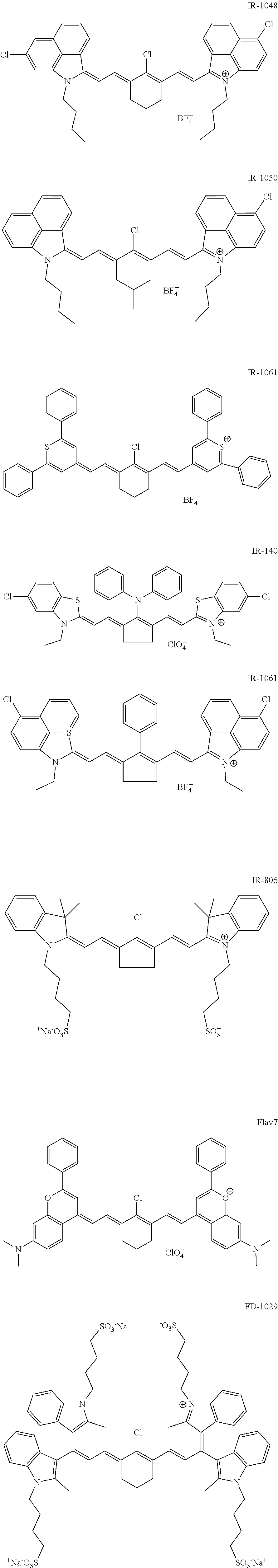

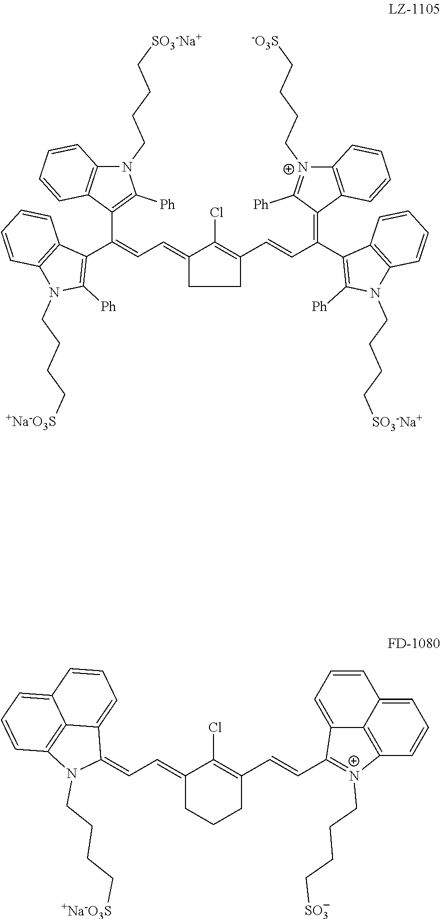

2. The compound of claim 1, wherein the pharmaceutically acceptable salt selected from the group consisting of sodium, potassium, ammonium, calcium, magnesium, lithium, cholinate, lysinium, and hydrogen salt.

3-6. (canceled)

7. A composition comprising a compound of claim 1 and a pharmaceutically acceptable carrier, excipient, or diluent wherein the composition comprises a pharmaceutically or therapeutically acceptable amount of the compound.

8-13. (canceled)

14. A method of optical or diagnostic imagining of a biological tissue that expresses a folate receptor, Glutamate carboxypeptidase II, prostate-specific membrane antigen (PSMA), carbonic anhydrase IX (CA IX), Fibroblast activation protein alpha, Glucose transporter 1, or cholecystokinin-2, the method comprising:
(a) contacting the biological tissue of a subject with a compound of claim 1;
(b) allowing time for the compound to distribute within the biological tissue;
(c) illuminating the biological tissue with an excitation wavelength absorbable by the compound; and
(d) detecting an optical signal emitted by the compound.

15. The method of claim 14, wherein the optical signal emitted by the compound is used to construct an image.

16. The method of claim 14, wherein diagnostic imaging is fluorescence-guided surgery or image-guided surgery.

17. The method of claim 14, wherein the biological tissue is selected from the group consisting of diseased tissue, abnormal tissue, tumor lesions, lymph nodes with metastatic tumor cells, cancer cells, circulating tumor cells (CTCs), and combinations thereof.

18. The method of claim 14, wherein the diagnostic imagining further comprises:
(e) diagnosing the subject with cancer, cardiovascular disease, neurodegenerative disease, immunologic disease, autoimmune disease, respiratory disease, metabolic disease, inherited disease, infectious disease, bone disease, and environmental disease.

19. (canceled)

20. The method of claim 17, wherein cancer cells or CTCs are selected from the group consisting of prostate cancer cells, cervical cancer cells, ovarian cancer cells, endometrial cancer cells, brain cancer cells, breast cancer cells, leukemia cells, kidney cancer cells, head and neck cancer cells, esophageal cancer cells, liver cancer cells, and lung cancer cells.

21. The method of claim 14, wherein the methods employ bioluminescence resonance energy transfer (BRET) or two-step fluorescence resonance energy transfer (FRET).

22. The method of claim 14, wherein the compound is used in a multifunctional imaging technique.

23. The method of claim 17, wherein biological tissue is CTCs and the method is used for (a) real-time monitoring, screening, and management of a subject having a disease:
(b) to determine the likelihood of the recurrence or remission of a disease in a subject; or
(c) to determine the likelihood of response to surgical treatment, chemotherapy, immunotherapy, radiotherapy, hormonal therapy.

24-30. (canceled)

31. The method of claim 14, wherein the subject has cancer and the cancer is selected from the group consisting of early-stage cancer, metastatic cancer, pancreatic cancer, gastrointestinal cancer, stomach cancer, colon cancer, ovarian cancer, cervical cancer, prostate cancer, glioma cancer, carcinoid cancer, thyroid cancer, lung cancer, bladder cancer, liver cancer, kidney cancer, sarcoma, breast cancer, brain cancer, testicular cancer, and melanoma.

32. (canceled)

33. (canceled)

34. The method of claim 14, wherein the compound is in contact with the biological tissue for at least about 30 minutes.

35. The method of claim 14, wherein the method is performed in vitro, in vivo, or ex vivo.

36. The method of claim 35, wherein the method is performed in vivo and the biological tissue is detected using two-photon microscopy, epifluorescence microscopic, or an innovative wearable.

37. The method of claim 36, wherein the innovative wearable is a smartwatch, a wrist band, earpiece, wearable microscope, or bicep band.

38. The method of claim 37, wherein the innovative wearable is a smartwatch, wherein the smartwatch employs sensors and algorithms for detecting and quantifying a subject's CTC levels.

39. (canceled)

40. The method of claim 37, wherein the innovative wearable is a wearable microscope, wherein the wearable microscope employs lasers to generate a fluorescent image.

41-43. (canceled)

44. The method of claim 17, wherein the CTCs are further isolated and/or enriched using ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods.

45. (canceled)

46. (canceled)

47. The compound of claim 1, wherein the NIR-II dye has an excitation and emission spectra in the second region near infrared.

48. The compound of claim 1, wherein the NIR-II dye has an absorption and emission maxima between about 1000 nm and 1700 nm.
